(19) United States
(12) Patent Application Publication
(10) Pub. No.: US 2023/0302154 A1
(43) Pub. Date: Sep. 28, 2023

(54) NANODRUGS FOR TARGETED DRUG DELIVERY AND USE THEREOF

(71) Applicants: THE BOARD OF REGENTS OF THE UNIVERSITY OF TEXAS SYSTEM, Austin, TX (US); UNIVERSITY OF CONNECTICUT, Farmington, CT (US)

(72) Inventors: Upal ROY, Edinburg, TX (US); Mu-Ping NIEH, Farmington, CT (US)

(21) Appl. No.: 18/041,811

(22) PCT Filed: Aug. 19, 2021
(86) PCT No.: PCT/US2021/046708

§ 371 Date: Feb. 16, 2023

Related U.S. Application Data
(60) Provisional application No. 63/067,682, filed on Aug. 19, 2020.

(51) Int. Cl. A61K 47/69; A61K 47/24; A61K 31/536; A61K 31/675; A61P 31/18

(52) U.S. Cl. CPC A61K 47/6913; A61K 47/6925; A61K 47/24; A61K 31/536; A61K 31/675; A61P 31/18

(57) ABSTRACT

The present disclosure provides target nanodrugs comprising nanocarriers, such as nanodiscs and/or liposomes, encapsulating a therapeutic agent. The nanodrugs may be conjugated to a targeting antibody, such as for delivery of the nanodrug across the blood brain barrier. The nanodrugs may comprise anti-retroviral therapy. Further provided herein are methods for the treatment of a disease or disorder by administering the target nanodrugs, such as for the treatment of HIV.

This application claims the benefit of U.S. Provisional Patent Application No. 63/067,682, filed Aug. 19, 2020, the entirety of which is incorporated herein by reference.

### BACKGROUND

### 1. Field

The present invention relates generally to the field of molecular biology and medicine. More particularly, it concerns methods and compositions for targeted delivery of drugs using nanocarriers.

### 2. Description of Related Art

Human Immunodeficiency Virus (HIV) continues to exist today as one of the deadliest viruses worldwide. While antiretroviral therapy exists to help decrease the overall disease mortality life and preserve essential life functions, the therapy is not a system-wide solution. Furthermore, current treatment options have other obstacles in terms of equal patient care including but not limited to cost of medication, and transmigration of the drugs across the Blood Brain Barrier (BBB). In particular, crossing the BBB is currently a major hindrance in the complete elimination of the virus residing within the brain. Promising studies which take advantage of nanotechnology has opened a door to new possibilities in the world of HIV treatment.

Nanoparticles (NPs) have been widely used as biodiagnostic and pharmaceutical carriers to enhance the efficacy of different applications. To date, three generations of NPs have been engineered for biomedical applications. The first generation of NPs was designed based on biocompatibility, water solubility, size and charge density to enhance cellular uptake and toxicity (Best et al., 2009; Varatharajan and Thomas, 2009). The second-generation NPs are equipped with two important features: stealth and active targeting. The third generation shifted the paradigm of design to new shapes, more complicated structures, and “intelligent” platforms that can release their payload on-demand (by environment or external stimuli) and have therapeutic/diagnostic (theranostic) properties (Overall et al., 2009).

In regards to cancer treatment or diagnosis, several important properties of NPs are desirable such as high biocompatibility, long in vivo circulation half-life, high accumulation at cancer tissues and high loading drug/diagnostic capacity. Significant attention has been drawn to a variety of nanocarriers, such as liposomes (Roy et al., 2013), solid lipid NPs (Suri et al., 2007), polymeric micelles (Sarkar et al., 2014), dendrimers (Liu et al., 2017), oil bodies (Aresh et al., 2016), aptamers (Walsh et al., 2014), and nanoporous lipid bilayers (WHO, 2013), demonstrating great potential for in vivo applications. That is due to their large drug carrying capacity, passive accumulation in malignant and inflamed tissues, long circulation times, and adaptability to multiple functions (Tahmasbi et al., 2019).

However, the HIV virus still persists at different pharmacological reservoirs of treated patients like the brain. Thus, there is an unmet need for an efficient drug carrier to reach those reservoir organs to control viral replication on site with drugs, such as a nanoparticle formulation that allows for the successful crossing of the BBB and the controlled release of antiretroviral drugs to further improve the lives of HIV infected patients.

### SUMMARY

In certain embodiments, the present disclosure targeted nanodrug composition comprising nanocarriers encapsulating a therapeutic agent, wherein the nanocarriers are conjugated to a targeting antibody or fragment thereof. In some aspects, the nanocarriers are nanodiscs and/or liposomes. In some aspects, the nanocarriers are “nanodiscs” which are low-polydispersity, spontaneously-forming discoidal bicelles composed of long- and short-chain lipids which encapsulate a therapeutic agent to form “nanodrugs”. In one embodiment, there is provided a targeted nanodrug composition comprising nanodiscs encapsulating a therapeutic agent, wherein the nanodiscs are conjugated to a targeting antibody. In some aspects, the nanocarriers are liposomes. In certain aspects, the nanocarriers are nanodiscs and liposomes.

In some aspects, the nanodiscs comprise a mixture of at least one long-chain phospholipid and one short-chain phospholipid. In certain aspects, the long-chain phospholipid is selected from the group consisting of dipalmitoyl phosphocholine (DPPC), dipalmitoyl phosphatidylglycerol (DPPG), dimyristoyl phosphatidylcholine (DMPC), dioleoylphosphatidylserine (DOPS), dioleoylphosphatidylglycerol (DOPG), dioleoylphosphatidylinositol (DOPI), dioleoylphosphatidic acid (DOPA), or a mixture thereof. In particular aspects, the long-chain phospholipid is DMPC. In specific aspects, the short-chain phospholipid is DPPC or dihexanoyl phosphatidylcholine (DHPC). In some aspects, the nanodisc further comprises an anionic phospholipid, such as 1,2-Dimyristoyl-sn-glycero-3-phosphoglycerol (DMPG). In particular aspects, the nanodiscs comprise [DPPG]/[DPPC] at a molar ratio of 0.01 to 0.1, such as 0.02, 0.03, 0.04, 0.05, 0.06, 0.07, 0.08, or 0.09 (e.g., 0.01-0.02, 0.02-0.03, 0.03-0.04, 0.04-0.05, 0.05-0.06, 0.06-0.07, 0.07-0.08, 0.08-0.09, or 0.09-0.1). In some aspects, the nanodiscs comprise ([DPPC]+[DPPG])/[DHPC] at a molar ratio of 2 to 5, such as 2-2.5, 2.5-3, 3-3.5, 3.5-4, 4-4.5, or 4.5-5 (e.g., 2, 2.5, 3, 3.5, 4, 4.5, or 5). In some aspects, the nanodrug is PEGylated. In some aspects, the nanodiscs further comprise polyethylene glycol (PEG2000)-conjugated distearoyl phosphoethanolamine (DSPE-PEG2000). In certain aspects, the nanodiscs comprise DPPC, DHPC, DPPG, and DSPE-PEG2000.

In certain aspects, the nanodiscs have a diameter of 30-40 nm, such as 31, 32, 33, 34, 35, 36, 37, 38, 39, or 40 nm. In particular aspects, the nanodiscs have a thickness of about 5 nm. In certain aspects, the nanodiscs have a hydrodynamic radius of 8-15 nm, such as 8, 9, 10, 11, 12, 13, 14, or 15 nm. In some aspects, the nanodiscs have a hydrodynamic radius of 5-15 nm, such as 10-13 nm, such as about 10, 11, 12, or 13 nm.

In some aspects, the liposomes comprise dipalmitoyl phosphocholine (DPPC), dipalmitoyl phosphatidylglycerol (DPPG), dimyristoyl phosphatidylcholine (DMPC), dioleoylphosphatidylserine (DOPS), dioleoylphosphatidylglycerol (DOPG), dioleoylphosphatidylinositol (DOPI), dioleoylphosphatidic acid (DOPA), dihexanoyl phosphatidylcholine (DHPC), or a mixture thereof. In certain aspects, the liposomes comprise DPPC and DPPG. In some aspects, the liposomes have a hydrodynamic radius of about 50 nm to about 500 nm, such as 300 nm to 500 nm, such as 350, 375, 400, 425, 450, 475, or 500 nm. In certain aspects, the liposomes have a lipid charge density of 1% to 10%, such as about 2% to 5%, such as 2%, 3%, 4%, or 5%.

In some aspects, the therapeutic agent is at least one anti-retroviral therapy, such as a protease inhibitor or reverse transcriptase inhibitor. In some aspects, the anti-retroviral therapy comprises tenofovir, efavirenz, lopinavir, ritonavir, emtricitabine, rilpivirine, tenofovir disoproxil fumarate, elvitegravir, cobicistat, elvitegravir, doravirine, lamivudine, dolutegravir, rilpivirine, bictegravir, atazanavir, abacavir, fostemsavir, raltegravir, maraviroc, enfuvirtide, enfuvirtide, tipranavir, fosamprenavir, darunavir, rilpivirine, nevirapine, etravirine, doravirine, and/or dultagravir. In certain aspects, the protease inhibitor is CRIXIVAN® (indinavir sulfate ethanolate or IDV), saquinavir, Invirase® (saquinavir mesylate or SQV), NORVIR® (ritonavir or RTV), VIRACEPT® (nelfinavir mesylate or NFV), LOPINAVIR (LPV), PROZEI® (amprenavir or APV), or REYATAZ® (atazanavir or ATV). In specific aspects, the reverse transcriptase inhibitor is Tenofovir (PMPA or TFV), RETROVIR® (zidovudine or AZT), EPIVIR® (lamivudine or 3TC), ZERIT® (sanilvudine or d4T), VIDEX® (didanosine or ddI), ZIAGEN® (abacavir sulfate or ABC), VIRAIVIUNE® (nevirapine or NVP), STOCRIN® (efavirenz or EFV), or RESCRIPTOR® (delavirdine mesylate or DLV). In some aspects, the anti-retroviral therapy comprises Tenofovir, Efavirenz, or lopinavir. In particular aspects, the anti-retroviral therapy does not comprise Efavirenz.

In some aspects, the nanodrug comprises a therapeutic agent to lipid ratio of 1:1 to 1:1000, such as 1:1 to 1:100, such as 1:2, 1:3, 1:4, 1:5, 1:6, 1:7, 1:8, 1:9, 1:10, 1:15, 1:20, or 1:25. In particular aspects, the nanodrug has a therapeutic agent to lipid ratio of about 1:3 to about 1:5.

In particular aspects, the targeting antibody or fragment thereof allows delivery to the brain, lymph nodes, and/or gut-associated lymphoid tissue. In some aspects, the targeting antibody or fragment thereof allows delivery across the blood brain barrier. the targeting antibody is neuron specific or microglia specific. In specific aspects, the microglia targeting antibody or fragment thereof is a Tmem19 antibody or a Siglec-H antibody. In some aspects, the targeting antibody or fragment thereof is anti-EGFR monoclonal antibody, or anti-IGFBP7 sdAb.

A further embodiment provides a pharmaceutical composition comprising a plurality of targeted nanodrugs of the present embodiments or aspects thereof (e.g., a targeted nanodrug composition comprising nanodiscs and/or liposomes encapsulating a therapeutic agent, wherein the nanodiscs and/or liposomes are conjugated to a targeting antibody or fragment thereof) in combination with a pharmaceutically acceptable carrier.

Another embodiment provides a method of delivering a therapeutic agent into a cell comprising administering an effective amount of targeted nanodrugs of the present embodiments or aspects thereof (e.g., a targeted nanodrug composition comprising nanodiscs and/or liposomes encapsulating a therapeutic agent, wherein the nanodiscs and/or liposomes are conjugated to a targeting antibody or fragment thereof) to the cell.

In yet another embodiment, there is provided a method of treating a disease or disorder in subject in need thereof comprising administering an effective amount of targeted nanodrugs of the present embodiments or aspects thereof (e.g., a targeted nanodrug composition comprising nanodiscs and/or liposomes encapsulating a therapeutic agent, wherein the nanodiscs and/or liposomes are conjugated to a targeting antibody or fragment thereof) to the subject. In some aspects, the subject is human.

In some aspects, the subject is positive for human immunodeficiency virus (HIV). In certain aspects, the subject has been previously treated with anti-retroviral therapy.

In certain aspects, the targeted nanodrug are administered orally, topically, intravenously, intraperitoneally, intramuscularly, endoscopically, percutaneously, subcutaneously, regionally, or by direct injection.

In some aspects, the method further comprises administering an additional therapeutic agent.

Another embodiment provides a pharmaceutical composition comprising targeted nanodrugs of the present embodiments or aspects thereof (e.g., a targeted nanodrug composition comprising nanodiscs and/or liposomes encapsulating a therapeutic agent, wherein the nanodiscs and/or liposomes are conjugated to a targeting antibody or fragment thereof) for use in the treatment of a disease or disorder in a subject. In some aspects, the subject is positive for HIV.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: (FIG. 1A) The Nanodisc structure is modeled here on the left half of the figure. Nanodiscs can comprise short-chain and long-chain lipids and may be further stabilized by PEGylated lipids (bottom). NDs measure at ˜30 nm in diameter and ˜5 nm in thickness. Their structure allows for additional surface/ligand modification. This lipid-based formulation can encapsulate the antiretroviral therapy drugs in the center of the structure (right). (FIG. 1B) Schematic and microscopic observation of nanodisc with drug (Mahabir et al., 2013).

FIG. 2: Molecular Surface Lipophilicity Potentials (MLSP) modeling results for Efavirenz (top), Tenofovir (middle) and Lopinavir (bottom). The red/orange color range are low lipophilic locations and violet/blue are high lipophilic locations in the molecule. MSLP for each is shown. The color ramp for the MLSP ranges from deep red color, representing lower lipophilic potential (LP), to the navy/violet color, representing higher LP. This analysis can provide LP surrounding each atom or group of atoms and the 3D spatial features of the molecular interactions in crystal. Hydrophilic and hydrophobic moieties of each drug were traced using MLSP of the molecule, which shows sites of high and low LP for Efavirenz and Tenofovir, respectively. These lipophilicity analyses explain why some molecules entrap easily into the bilayer and some do not.

FIG. 3: Small angle X-ray scattering (SAXS) was used to provide the detailed discoidal core-shell architecture of the designed nanoparticles. This technique is very sensitive to the electron density distribution in the structure of nanoassemblies averaged in time. The pattern x-axis is 1/Angstrom. So higher x-values correlate to smaller distances. The lipid bilayers are a lipophilic core (hydrocarbon chains) sandwiched by two shells (hydrophilic phosphatidylcholine head groups). Since the phosphate group has the highest electron density in the system—higher than those of hydrocarbon tails and water, the electron density profile across the bilayer (i.e., water-head group shell-hydrocarbon core-head group shell-water) can be approximated by a “square well”. As a result, the SAXS pattern leads to a broad peak corresponding to the correlation length of head group-head group distance as shown.

FIGS. 4A-4D: ROS Assay of Nanodisc formulation in SK-N-MC. The ROS assay was performed at different concentrations of the nanodisc formulation for non-PEGulated Efavirenz (FIG. 4A), PEGylated Efavirenz (FIG. 4B), non-PEGylated Tenofovir (FIG. 4C), PEGylated Tenofovir (FIG. 4D). The data is presented as RFU of each treatment. The ROS production was measured in nanodisc formulation treated cells and compared with untreated control. H₂O₂ treatment is the positive control and catalase is an antioxidant. The data is expressed in mean values of RFU. This is cumulative data of eight independent experiments indicating no significant change in ROS production with increasing concentration of nanodisc formulation.

FIGS. 5A-5D: Cytotoxicity of Nanodisc in SK-N-MC cells. These cells were treated with 0.1 mg/mL nanodisc formulation for 24 hours including non-PEGylated Efavirenz (FIG. 5A), PEGylated Efavirenz (FIG. 5B), non-PEGylated Tenofovir (FIG. 5C), and PEGylated Tenofovir (FIG. 5D). After incubation, MTS assays were performed and optical density was measured at 490 nm. The data is presented at percentage cell survival of nanodisc formulation. The control is at 100 percent viability. The difference in the concentrations had no significant effect on cell survival.

FIG. 6: Effect of nanodrug on ROS production in SK-N-MC cells. p<0.0001. Catalase (Cat.): Negative control; H₂O₂: Positive Control.

FIG. 7: HIV-LTR gene expression in human macrophages treated with unformulated TDF and nanodrug, p<0.0001.

FIG. 8: DLS of NDs at varying drug-to-lipid ratios. DLS was used to determine the size distribution within the solutions. Results demonstrated the hydrodynamic radius of the ND ranged from −10-13 nm and confirmed the uniformity of the NDs.

FIG. 9: SAXS graph of NDs at varying drug-to-lipid ratios. SAXS was used to provide discoidal morphology. Retention of the valley and broad bilayer peaks indicated that samples had retained their structures.

FIGS. 10A-10B: Cell viability assay of ND on HMC-3 and SH-SY5Y cells. A cell viability analysis of (FIG. 10A) HMC-3 and (FIG. 10B) SH-SY5Y cells when treated with ND at a drug-to-lipid ratios of 1:20, 1:4 and FD-TFV. Graphical representation of was made as cell viability percentage (%) at different TFV concentrations (0.01 mg/mL-0.1 mg/mL) of the formulations. Untreated (control) cells were considered 100% viability and % cell survival was monitored based on control. Cell viability % was measured as mean±SD of three independently replicated experiments. Statistical significance was calculated by two-way ANOVA test with posthoc Dunnett's multiple comparisons test. A value of p<0.05 was indicative of significance compared to the control (*).

FIGS. 11A-11B: Cell viability assay of empty ND on HMC-3 and SH-SY5Y cells. Graphical representation was made as cell viability percentage (%) varying lipid concentrations of the ND (0.01-0.53%) in HMC-3 cells (FIG. 11A) and SH-SY5Y cells (FIG. 11B). Untreated (control) cells were considered 100% viability, and % cell survival was monitored based on control. Cell viability % was measured as mean±SD of three independently replicated experiments. Statistical significance was calculated by one-way ANOVA test with posthoc Dunnett's multiple comparisons test. A value of p<0.05 was indicative of significance compared to the control (*).

FIGS. 12A-12B: Effect of ND at 1:20 and 1:4 and FD-TFV on ROS production on HMC-3 and SH-SY5Y cells. (FIG. 12A) HMC-3 and (FIG. 12B) SH-SY5Y cells were exposed at different concentrations (0.01 mg/mL-0.1 mg/mL). Graphical representation was made in ROS production; ROS production was measured in terms as mean±SD relative fluorescence units (RFU) of three independently replicated experiments. Statistical significance was calculated by two-way ANOVA test with posthoc Dunnett's multiple comparisons test. A value of p<0.05 was indicative of significance compared to untreated cells (*).

FIGS. 13A-13B: Effect of empty ND on ROS production on HMC-3 and SH-SY5Y cells. (FIG. 13A) HMC-3 and (FIG. 13B) SH-SY5Y cells were exposed at different lipid concentrations (0.01-0.1%). Graphical representation was made in ROS production; ROS production was measured in terms of mean±SD relative fluorescence units (RFU) of three independently replicated experiments. Statistical significance was calculated by one-way ANOVA test with posthoc Dunnett's multiple comparisons test. A value of p<0.05 was indicative of significance compared to untreated cells (*).

FIG. 14: Drug release profiles of ND (1:20 and 1:4) vs. FD-TFV in vitro. Graphical representation was made in terms of cumulative drug release percentage (%) and was measured in terms as mean %±SD of n=3. Statistical significance was calculated by two-way ANOVA test with posthoc Dunnett's multiple comparisons test. A value of p<0.05 was indicative of significance when compared to FD-TFV (*). Note: Some initial timepoints are not marked for significance due to overlapping in graph.

FIGS. 15A-15B: BBB model's TEER values and sustained drug release study from 1:20 and 1:4 ND in vitro; 1:20 ND, 1:4 ND and FD-TFV were introduced into the apical chamber of the BBB model. (FIG. 15A) TEER values of the BBB were measured to ensure formulations did not significantly affect the integrity of the BBB. (FIG. 15B) Drug release of formulations through the BBB was observed and measured up to 10 days. Graphical representation was made in terms of average drug release percentage (%) and was measured in terms as mean %±SD of n=3. Statistical significance was calculated by two-way ANOVA test with posthoc Dunnett's multiple comparisons test. A value of p<0.05 was indicative of significance when compared to FD-TFV (*).

FIG. 16: In vitro cellular uptake study of ND (1:4 and 1:20) and FD-TFV. Graphical representation was made in terms of average intracellular concentration (IC) and was measured in terms as mean±SD of n=3. Statistical significance was calculated by two-way ANOVA test with posthoc Dunnett's multiple comparisons test. A value of p<0.05 was indicative of significance when compared to FD-TFV (*).

FIG. 17: Plasma concentrations of TFV after a single iv dose to male and female BALB/c mice. Graphical representation was made in terms of plasma concentration (ng/mL). each data point represents the mean±SD of n=12 mice. FD-TFV treated females were found to be statistically significant compared to other treatment groups; however, this may due to an individual animal variation as one mouse had a higher plasma concentration at first time point than other mice in group. Statistical significance was calculated by two-way ANOVA test with posthoc Dunnett's multiple comparisons test. A value of p<0.05 was indicative of significance.

FIG. 18: Extended drug release profiles of liposome-TFV at varying drug-to-lipid concentrations. Liposomes had following drug-to-lipid ratios: Liposome 1 (1:2.3), Liposome 2 (1:3.7) and Liposome 3 (1:3.7). Graphical representation was made in terms of cumulative drug release percentage (%) and was measured in terms of mean %±SD. Statistical significance was calculated by two-way ANOVA test with posthoc Dunn's test. A value of p<0.05 was indicative of significance (*). Liposomes 1, 2 and 3 had statistically significant drug release up to 6 days compared to FD-TFV.

### DESCRIPTION OF ILLUSTRATIVE EMBODIMENTS

One of the challenges associated with nanoparticles is the human microenvironment. The endothelium in humans lack fenestrations and have dense collagen matrices in the extracellular matrix (ECM). Therefore, in most cases, a smaller nanocarrier is needed for human therapies. Larger particles can be prevented from diffusing from the matrix entirely, and smaller particles would be excreted through the renal system. Accordingly, in certain embodiments, the present disclosure provides nanocarriers for the delivery of therapeutic agents. The nanocarriers may be nanodiscs and/or liposomes. For example, hydrophilic therapeutic agents may be delivered in liposomes or in a mixture of liposomes and nanodiscs, the ratio of liposomes to nanodiscs may be variable.

In certain embodiments, the present disclosure provides “nanodiscs” which are low-polydispersity, spontaneously-forming discoidal bicelles composed of long- and short-chain lipids, such as dimyristoyl phosphatidylcholine (DMPC) and dihexanoyl phosphatidylcholine (DHPC). Nanocarriers, such as nanodiscs or liposomes, may encapsulate a therapeutic agent to form “nanodrugs”. The nanodiscs may be further stabilized by polyethylene glycol-conjugated (PEGylated) lipids. The nanodrugs may have a diameter of about 7-50 nm, such as about 20-50 nm (e.g., about 20-25, 25-30, 30-35, 35-40, 40-54, or 45-50 nm, particularly about 30-40 nm (e.g., about 30 nm), and a thickness of about 3-10 nm, such as about 5 nm. In particular aspects, the present nanodrugs have uniform dimensions and can be modified to have a desired surface charge, such as a negative surface charge for longer blood half-life. The structure of the nanodrug may be optimal for a lipid-based drug delivery system which will help preserve the drug in the formulation until it begins to be metabolized by the body.

In particular, the present nanodrugs can have increased uptake and more diverse mechanisms for endocytosis as well as faster diffusion across the extracellular matrix due to size and morphology resulting in increased rate of apoptosis in the target cells, thus reducing potential drug resistance. Moreover, the discoidal shape contributes to the extension of blood circulation time due to the ability to coordinate alignment with blood flow and avoid vascular filtration, collisions, and phagocytosis. In addition, the present nanodrugs can be produced at a large scale, such as for therapeutics and large-scale nanomanufacturing of the NPs.

In some embodiments, nanodrugs are provided which encapsulate an anti-retroviral drug for the treatment of HIV. Further provided herein are methods of manufacturing and developing the nanodrug formulation, such as nanodrugs comprising an anti-HIV drug and a targeting antibody. For example, the anti-HIV drug may be tenofovir. Tenofovir (TFV) is an acyclic nucleotide analogue with a potent in vitro and in vivo antiretroviral activity; however, tenofovir has limited oral bioavailability in animals. TFV is a widely used antiretroviral drug belonging to the NRTI drug class as it inhibits HIV-1 and HIV-2 DNA polymerases (reverse transcriptase) along with other viral DNA polymerases at the third main step of HIV-1's replication cycle. TFV's inhibition results in DNA chain termination the impairment of viral replication. TFV exists as a dianion compound which makes it very polar. By being such a polar species, TFV does not readily undergo passive diffusion across cellular membranes, resulting in low bioavailability after oral administration. TFV has been found to cross the blood-cerebrospinal fluid barrier but cannot readily cross the BBB to reach deep brain sites due to its hydrophilic nature. Therefore, the present studies looked into a nanodisc based drug delivery that could be used as a potential method to deliver extended-release of TFV for long-term inhibition of HIV-1 within the brain. The nanodiscs were developed and characterized by TFV encapsulated within the nanodisc structure. Two different drug-to-lipid ratios of 1:20 ND and 1:4 nanodiscs were observed for their biological safety sustained-release properties.

The nanodrug can comprise a targeting antibody which allows delivery across the BBB. The targeting antibody may be a neuron or microglia specific antibody, or a neurofilament antibody. In some aspects, the targeting antibody is anti-Tmem19 antibody or anti-Siglec-H antibody, such as for targeting the brain. The nanodrug may be taken up by macrophages which naturally cross the BBB. In some aspects, M-cell protein (e.g., and anti-GP2 antibody) may be used for targeting gut lymphoid tissue. While inside the brain, the macrophages can travel to the site of inflammation as a natural immune response. At the site of neuronal inflammation where NF level is relatively higher, the released nanodrug will potentially bind with the free NF protein present in that microenvironment and release the drug onsite from ND through a desorption mechanism. The released drug can directly act on HIV-1 released from activated microglia (reservoir for HIV) present on that site. Thus, the nanodrug can slowly release its contents to the affected region of the brain where the HIV virus resides in small yet therapeutically effective, controlled dosages over an extended period of time. The slow release of the drug is made possible due to the structure of the nanodrug allowing for drug viable for an extended period of time. In addition, the nanodrug structure itself allows for slow degradation over an extended period of time, aiding in the slow release of the drug. The present studies showed that the anti-HIV drug encapsulated nanodrug was non-toxic to neuronal cells and significantly more effective then unformulated drugs. Thus, a treatment strategy is provided where the patient will be given a less frequent dose of drugs instead of daily dosing as recommended in current therapy with a significantly higher therapeutic efficacy.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” means, in general, the stated value plus or minus 5%.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “therapeutic agent” as used herein refers to any agent that can be administered to a subject for the purpose of obtaining a therapeutic benefit of a disease or health-related condition. For example, nanodrugs that include a therapeutic agent may be administered to a subject for the purpose of reducing the size of a tumor, or reducing or inhibiting viral replication.

A “diagnostic agent” as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

The terms “lipid” and “phospholipid” are used interchangeably and to refer to structures containing lipids, phospholipids, or derivatives thereof comprising a variety of different structural arrangements which lipids are known to adopt in aqueous suspension. These structures include, but are not limited to, lipid bilayer vesicles, micelles, bicelles, liposomes, emulsions, vesicles, lipid ribbons or sheets. The lipids may be used alone or in any combination which one skilled in the art would appreciate to provide the characteristics desired for a particular application. In addition, the technical aspects of lipid constructs and liposome formation are well known in the art and any of the methods commonly practiced in the field may be used for the present disclosure.

The term “long chain lipid” or “long chain phospholipid” refers to lipids having a carbon chain length of about 13, 14, 15, 16, 17, 18, 19, 20, 21, 22, 23 or 24. In one embodiment, the chain length is selected from a chain length of 18, 19, or 20. Representative examples of long chain lipids that may be used include, but are not limited to the following lipids: dimyristoyl phosphatidylcholine (DMPC), 14:0 PS 1,2-Dimyristoyl-sn-Glycero-3-[Phospho-L-Serine] (Sodium Salt) (DMPS); 16:0 PS 1,2-Dipalmitoyl-sn-Glycero-3-[Phospho-L-Serine] (Sodium Salt) (DPPS); 17:0 PS 1,2-Diheptadecanoyl-sn-Glycero-3-[Phospho-L-Serine] (Sodium Salt); 18:0 PS 1,2-Distearoyl-sn-Glycero-3-[Phospho-L-Serine] (Sodium Salt) (DSPS); 18:1 PS 1,2-Dioleoyl-sn-Glycero-3-[Phospho-L-Serine] (Sodium Salt) (DOPS); 18:2 PS 1,2-Dilinoleoyl-sn-Glycero-3-[Phospho-L-Serine] (Sodium Salt); 20:4 PS 1,2-Diarachidonoyl-sn-Glycero-3-[Phospho-L-Serine] (Sodium Salt); 22:6 PS 1,2-Didocosahexaenoyl-sn-Glycero-3-[Phospho-L-Serine] (Sodium Salt); 16:0-18:1 PS 1-Palmitoyl-2-Oleoyl-sn-Glycero-3-[Phospho-L-Serine] (Sodium Salt) (POPS); 16:0-18:2 PS 1-Palmitoyl-2-Linoleoyl-sn-Glycero-3-[Phospho-L-Serine] (Sodium Salt); 16:0-22:6 PS 1-Palmitoyl-2-Docosahexaenoyl-sn-Glycero-3-[Phospho-L-Serine] (Sodium Salt); 18:0-18:1 PS 1-Stearoyl-2-Oleoyl-sn-Glycero-3-[Phospho-L-Serine] (Sodium Salt); 18:0-18:2 PS 1-Stearoyl-2-Linoleoyl-sn-Glycero-3-[Phospho-L-Serine] (Sodium Salt); 18:0-20:4 PS 1-Stearoyl-2-Arachidonoyl-sn-Glycero-3-[Phospho-L-Serine] (Sodium Salt); 18:0-22:6 PS 1-Stearoyl-2-Docosahexaenoyl-sn-Glycero-3-[Phospho-L-Serine] (Sodium Salt); 16:0 PC 1,2-Dipalmitoyl-sn-Glycero-3-Phosphocholine (DPPC); 17:0 PC 1,2-Diheptadecanoyl-sn-Glycero-3-Phosphocholine; 18:0 PC 1,2-Distearoyl-sn-Glycero-3-Phosphocholine (DSPC); 16:1 PC (Cis) 1,2-Dipalmitoleoyl-sn-Glycero-3-Phosphocholine; 16:1 Trans PC 1,2-Dipalmitelaidoyl-sn-Glycero-3-Phosphocholine; 18:1 PC Delta6 (cis) 1,2-Dipetroselinoyl-sn-Glycero-3-Phosphocholine; 18:2 PC (cis) 1,2-Dilinoleoyl-sn-Glycero-3-Phosphocholine; 18:3 PC (cis) 1,2-Dilinolenoyl-sn-Glycero-3-Phosphocholine; 20:1 PC (cis) 1,2-Dieicosenoyl-sn-Glycero-3-Phosphocholine; 22:1 PC (cis) 1,2-Dierucoyl-sn-Glycero-3-Phosphocholine; 22:0 PC 1,2-Dibehenoyl-sn-Glycero-3-Phosphocholine; 24:1 PC (cis) 1,2-Dinervonoyl-sn-Glycero-3-Phosphocholine; 16:0-18:0 PC 1-Palmitoyl-2-Stearoyl-sn-Glycero-3-Phosphocholine; 16:0-18:1 PC 1-Palmitoyl-2-Oleoyl-sn-Glycero-3-Phosphocholine; 16:0-18:2 PC 1-Palmitoyl-2-Linoleoyl-sn-Glycero-3-Phosphocholine; 18:0-18:1 PC 1-Stearoyl-2-Oleoyl-sn-Glycero-3-Phosphocholine; 18:0-18:2 PC 1-Stearoyl-2-Linoleoyl-sn-Glycero-3-Phosphocholine; 18:1-18:0 PC 1-Oleoyl-2-Stearoyl-sn-Glycero-3-Phosphocholine; 18:1-16:0 PC 1-Oleoyl-2-Palmitoyl-sn-Glycero-3-Phosphocholine; 18:0-20:4 PC 1-Stearoyl-2-Arachidonyl-sn-Glycero-3-Phosphocholine; 16:0-18:1 PG 1-Palmitoyl-2-Oleoyl-sn-Glycero-3-[Phospho-rac-(1-glycerol)] (Sodium Salt) (POPG); 18:1 PG 1,2-Dioleoyl-sn-Glycero-3-[Phospho-rac-(1-glycerol)] (Sodium Salt) (DOPG); 18:1 PA 1,2-Dioleoyl-sn-Glycero-3-Phosphate (Monosodium Salt) (DOPA); 18:1 PI 1,2-Dioleoyl-sn-Glycero-3-Phosphoinositol (Ammonium Salt); 16:0 (D31)-18:1 PI 1-Palmitoyl(D31)-2-Oleoyl-sn-Glycero-3-Phosphoinositol (Ammonium Salt); 18:1 PE 1,2-Dioleoyl-sn-Glycero-3-Phosphoethanolamine (DOPE); 18:2 PE 1,2-Dilinoleoyl-sn-Glycero-3-Phosphoethanolamine.

The term “short chain lipid” or “short chain phospholipid” refers to lipids having a carbon chain length of 4, 5, 6, 7, 8, 9, 10, 11 or 12. In one embodiment, the carbon chain length is 6, 7, 8 9 or 10. In one embodiment, the carbon chain length is 6, 7 or 8. Examples of negative short chain lipids are available at the website www.avantilipids.com. Examples of short chain lipids that may be used with the present invention include, but are not limited to, the following: 06:0 PS (DHPS) 1,2-Dihexanoyl-sn-Glycero-3-[Phospho-L-Serine] (Sodium Salt); 08:0 PS 1,2-Dioctanoyl-sn-Glycero-3-[Phospho-L-Serine] (Sodium Salt); 03:0 PC 1,2-Dipropionoyl-sn-Glycero-3-Phosphocholine; 04:0 PC 1,2-Dibutyroyl-sn-Glycero-3-Phosphocholine; 05:0 PC 1,2-Divaleroyl-sn-Glycero-3-Phosphocholine; 06:0 PC (DHPC) 1,2-Dihexanoyl-sn-Glycero-3-Phosphocholine; 07:0 PC 1,2-Diheptanoyl-sn-Glycero-3-Phosphocholine; 08:0 PC 1,2-Dioctanoyl-sn-Glycero-3-Phosphocholine; 09:0 PC 1,2-Dinonanoyl-sn-Glycero-3-Phosphocholine; 06:0 PG 1,2-Dihexanoyl-sn-Glycero-3-[Phospho-rac-(1-glycerol)] (Sodium Salt); 08:0 PG 1,2-Dioctanoyl-sn-Glycero-3-[Phospho-rac-(1-glycerol)] (Sodium Salt); 06:0 PA 1,2-Dihexanoyl-sn-Glycero-3-Phosphate (Monosodium Salt); 08:0 PA 1,2-Dioctanoyl-sn-Glycero-3-Phosphate (Monosodium Salt); 06:0 PE 1,2-Dihexanoyl-sn-Glycero-3-Phosphoethanolamine; 08:0 PE 1,2-Dioctanoyl-sn-Glycero-3-Phosphoethanolamine.

As used herein, the term “spontaneously formed” is intended to encompass the meaning known in the art, wherein the formation of the liposome requires the application of minimal or no mechanical force to the mixture of phospholipids, though it is to be understood that the application of mechanical force, such as via vortexing or mixing, may be applied to facilitate formation of the liposome composition.

The terms “stable” or “stabilized”, as used herein, means that the nanodrugs may be substantially resistant to degradation, including, for example, loss of vesicle structure or encapsulated gas or gaseous precursor, for a useful period of time. Typically, the nanodrugs employed in the present description have a desirable shelf life, often retaining at least about 90% by volume of its original structure for a period of at least about two to three weeks under normal ambient conditions. In preferred form, the nanodrugs are desirably stable for a period of time of at least about 1 month, more preferably at least about 2 months, even more preferably at least about 6 months, still more preferably about eighteen months, and yet more preferably up to about 3 years.

The term “encapsulated” refers to the active agent being enveloped within a phospholipid membrane, such that the active agent is protected from the outside environment.

The terms “anti-retroviral drug” and “substance having anti-retroviral activity” as used herein are interchangeable and refer to any agent such as a chemotherapeutic, peptide, antibody, antisense, ribozyme, vaccine, immunostimulants such as interferon, a nucleoside or non-nucleoside reverse transcriptase inhibitor, protease inhibitor, integrase inhibitor, inhibitor of binding between a host cell receptor (e.g., CD4, CXCR4, CCR5) and a retrovirus, or any combination thereof, that is capable of inhibiting retrovirus replication or cytopathogenicity. Thus, an “anti-HIV drug” or a “substance having anti-HIV activity” as used herein refers to an anti-retroviral drug that is capable of inhibiting HIV replication or cytopathogenicity. As used herein, “inhibiting” refers to the decrease or cessation of at least one activity or characteristic associated with a virus, protein, enzyme, or any other compound.

### II. TARGETED NANOCARRIERS

A. Nanodiscs

The nanodiscs of the present disclosure may be produced by mixing long-chain lipids and short-chain lipids, such as zwitterionic lipids, to spontaneously form discoidal bicelles (bilayered micelles) after the addition of an aqueous solution. The long-chain fatty acids may comprise dimyristoyl phosphatidylcholine (DMPC). Short-chain fatty acids may comprise dihexanoyl phosphatidylcholine (DHPC). The mixture may further comprise an anionic lipid, such as dimyristoyl phosphatidylglycerol (DMPG), dioleoyl phosphatidylserine (DOPS), Dioleoylphosphatidyl-glycerol (DOPG), 1,2-dioleoyl-phosphatidyinositol (DOPI) or 1,2-dioleoylphosphatidic acid (DOPA). “Anionic phospholipids” refer to phospholipids having negative charge, including phosphate, sulphate and glycerol-based lipids. The lipids may be added at a molar ratio between 2 and 5 (e.g., 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9). In some aspects, the DMPC/DMPG/DHPC molar ratio is 5.0/0/5/15. The lipids may be mixed homogeneously via a temperature cycling between 10° C. and 50° C. In some aspects, the nanodiscs will form and have a diameter of about 30-40 nm and a thickness of about 5 nm.

In one exemplary method, a 15-25% (by weight) lipid mixture, such as 15%, 16%, 17%, 18%, 19%, 20%. 21%, 22%, 23%, 24%, or 25% by weight, is dispersed in 99.9% deuterium oxide (D20). The mixture may further comprise a salt, such as NaCl at a concentration of 1-5 nM, such as 1, 2, 3, 4, or 5 nM. The resulting nanodiscs may be stored at 4° C. The nanodiscs may be diluted, such as diluted to 0.1 to 20% by weight (e.g., 0.2, 0.3, 0.5, 1, 3, 5, 10, 15, or 20% by weight).

The targeting antibody can be conjugated to the lipid nanodisc if the lipid mixture contains 1-5 mole % of 1,2-distearoyl-sn-glycero-3-phosphoethanolamine-N-[amino(polyethylene glycol)-2000](ammonium salt) or 1,2-distearoyl-sn-glycero-3-phosphoethanolamine-N-[maleimide(polyethylene glycol)-2000] (ammonium salt), in targeting antibody containing IVIES buffer (0.1 M MES, 0.5 M NaCl, pH 5.5) using Sulfo-NHS and EDC in sequence (Iqbal et al., 2011; incorporated herein by reference).

The nanodrugs of the present disclosure may comprise a therapeutic and/or diagnostic agent, such as in methods for diagnosing the presence or absence of a disease in a patient and/or in methods for the treatment of disease in a patient. The active agents may be neutral, positively or negatively charged. Examples of suitable active agents include diagnostic agents, pharmaceuticals, drugs, synthetic organic molecules, proteins, peptides, vitamins, steroids and genetic material, including nucleosides, nucleotides and polynucleotides.

In some embodiments, the present nanodrugs may be loaded with analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); anti asthamati c s (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives); antifungal agents (e.g., griseofulvin, ketoconazole, itraconizole, amphotericin B, nystatin, and candicidin); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propanolol, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, and); agents useful for calcium regulation (e.g., calcitonin, and parathyroid hormone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., anti-viral drugs, anti-viral molecule/s (antibody), interferon alpha, beta or gamma, zidovudine, amantadine hydrochloride, ribavirin, acyclovir and any other synthesized products); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate; steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, E, K, and the like); as well as other drugs such as mitotane, halonitrosoureas, anthrocyclines, and ellipticine.

In particular embodiments, the present nanodrugs are loaded with an anti-retroviral therapy, such as for the treatment of HIV. The anti-retroviral therapy may include but is not limited to zidovudine or AZT (Retrovir), didanosine or DDI (Videx), stavudine or D4T (Zenith), lamivudine or 3TC (Epivir), zaicitabine or DDC (Hivid), abacavir sulphate (Ziagen), tenofovir disoproxil fumarate (Viread), emtricitabine (Emtriva), Combivir (contains 3TC and AZT), Trizivir (contains abacavir, 3TC and AZT), Epzicorm (contains abacavir and lamivudine); nevirapine (Viramune), delavirdine (Rescriptor), efavirenz (Sustiva), saquinavir (Invirase, Fortovase), indinavir (Crixivan), ritonavir (Norvir), nelfinavir (Viracept), amprenavir (Agenerase), atazanavir (Reyataz), Evotaz (contains atazanavir and cobicistat), fosamprenavir (Lexiva), Kaletra (contains lopinavir and ritonavir), enfuvirtide (T-20, Fuzeon), Truvada (contains Tenofovir and Emtricitabine), darunavir (Prezista), Prezcobix (contains darunavir and cobicistat), dolutegravir (Tivicay), Triumeq (contains dolutegravir, abacavir and lamivudine), elvitegravir (Vitekta), Genvoya (contains elvitegravir, cobicistat, tenofovir alafenamide fumarte and emtricitabine), Stribild (contains elvitegravir, cobicistat, tenofovir disoproxil fumarte and emtricitabine) raltegravir (Isentress), Complera (contains emtricitabine, tenofovir disoproxil fumarte, rilpivirine) and Atripla (contains fixed-dose triple combination of tenofovir, emtricitabine and efavirenz).

Examples of a HIV reverse transcriptase inhibitor include, but are not limited to, Retrovir® (zidovudine or AZT), Epivir® (lamivudine or 3TC), Zerit® (sanilvudine or d4T), Videx® (didanosine or ddI), Hivid® (zalcitabine), Ziagen® (abacavir sulfate or ABC), Viramune® (nevirapine or NVP), Stocrin® (efavirenz or EFV), Rescriptor® (delavirdine mesylate or DLV), Combivir® (zidovudine+lamivudine), Trizivir® (abacavir sulfate+lamivudine+zidovudine), Coactinon® (emivirine), Phosphonovir®, Coviracil®, alovudine (3′-fluoro-3′-deoxythymidine), Thiovir (thiophosphonoformic acid), Capravirin (5-[(3,5-dichlorophenyl)thio]-4-isopropyl-1-(4-pyridylmethyl)imidazole-2-methanol carbamic acid), Tenofovir (PMPA or TFV), Tenofovir disoproxil fumarate ((R)-[[2-(6-amino-9H-purin-9-yl)-1-methyl ethoxy]methyl]phosphonic acid bis(isopropoxycarbonyloxymethyl)ester fumarate), DPC-083 ((4 S)-6-chloro-4-[(1E)-cyclopropylethenyl]-3,4-dihydro-4-trifluoromethyl-2(1H)-quinazolinone), DP C-961 ((4 S)-6-chloro-4-(cyclopropylethynyl)-3,4-dihydro-4-(trifluoromethyl)-2(1H)-quinazolinone), DAPD ((−)-β-D-2,6-diaminopurine dioxolane), Immunocal, MSK-055, MSA-254, MSH-143, NV-01, TMC-120, DPC-817, GS-7340, TMC-125, SPD-754, D-A4FC, capravirine, UC-781, emtricitabine, alovudine, Phosphazid, UC-781, BCH-1b618, DPC-083, Etravirine, BCH-13520, MIV-210, abacavir sulfate/lamivudine, GS-7340, GW-5634, GW-695634, Truvada® (tenofovir+emtricitabine), elvucitabine, GW 204937, and GW 678248.

Examples of a HIV protease inhibitor include, but are not limited to, Crixivan® (indinavir sulfate ethanolate or IDV), saquinavir, Invirase® (saquinavir mesylate or SQV), Norvir® (ritonavir or RTV), Viracept® (nelfinavir mesylate or NFV), lopinavir (LPV), Prozei® (amprenavir or APV), Kaletra® (ritonavir+lopinavir), mozenavir dimesylate ([4R-( 4α, 5α,6β)]-1-3-bis[(3-aminophenyl)methyl]hexahydro-5,6-dihydroxy-4,7-bis(phenylmethyl)-2H-1,3-diazepin-2-one dimethanesulfonate), tipranavir (TPV or 3′-[(1R)-1-[(6R)-5,6-dihydro-4-hydroxy-2-oxo-6-phenyl ethyl-6-propyl-2H-pyran-3-yl]propyl]-5-(trifluoromethyl)-2-pyridinesulfonamide), lasinavir (N-[5(S)-(tert-butoxycarbonylamino)-4(S)-hydroxy-6-phenyl-2(R)-(2,3,4-trimethoxybenzyl)hexanoyl]-L-valine 2-methoxyethylenamide), KNI-272 ((R)-N-tert-butyl-3-[(2S,3 S)-2-hydroxy-3-N-[(R)-2-N-(isoquinolin-5-yloxyacetyl)amino-3-methylthiopropanoyl]amino-4-phenylbutanoyl]-5,5-dimethyl-1,3-thiazolidine-4-carboxamide), GW-433908, TMC-126, DPC-681, buckminsterfullerene, MK-944A (MK944 (N-(2(R)-hydroxy-1(S)-indanyl)-2(R)-phenylmethyl-4 (S)-hydroxy-5-[4-(2-benzo[b]furanylmethyl)-2(S)-(tert-butylcarbamoyl)piperazin-1-yl]pentanamide)+indinavir sulfate), JE-2147 ([2(S)-oxo-4-phenylmethyl-3 (S)-[(2-methyl-3-oxy)phenylcarbonylamino]-1-oxabutyl]-4-[(2-methylphenyl)methylamino]carbonyl-4 (R)-5,5-dimethyl-1,3-thiazole), BMS-232632 ((3 S,8 S,9 S,12 S)-3,12-bis(1,1-dimethyl ethyl)-8-hydroxy-4,11-dioxo-9-(phenylmethyl)-6-[[4-(2-pyridinyl)phenyl]methyl]-2,5,6,10,13-pentaazatetradecanedicarboxylic acid dimethyl ester), DMP-850 ((4R,5 S,6 S,7R)-1-(3-amino-1H-indazol-5-ylmethyl)-4,7-dibenzyl-3-butyl-5,6-dihydroxyperhydro-1,3-diazepin-2-one), DMP-851, RO-0334649, Nar-DG-35, R-944, VX-385, TMC-114, Fosamprenavir sodium, Fosamprenavir calcium, Darunavir, GW-0385, R-944, RO-033-4649, AG-1859, and Reyataz® (atazanavir; ATV).

Other anti-HIV drugs include a DNA polymerase inhibitor or DNA synthesis inhibitor, exemplified by, but not limited to, Foscavir®, ACH-126443 (L-2′,3′-didehydro-dideoxy-5-fluorocytidine), entecavir ((1 S,3 S,4S)-9-[4-hydroxy-3-(hydroxymethyl)-2-methylenecyclopentyl]guanine), calanolide A ([10R-(10α, 11β,12α)]-11,12-dihydro-12-hydroxy-6,6,10,11-tetramethyl-4-propyl-2H,6H,10H-benzo[1,2-b:3,4-b′:5,6-b″]tripyran-2-one), calanolide B, NSC-674447 (1,1′-azobisformamide), Iscador (viscum album extract), and Rubutecan.

An HIV antisense drug is exemplified by, but not limited to, HGTV-43 and GEM-92. An anti-HIV antibody is exemplified by, but not limited to, NM-01, PRO-367, KD-247, CYTOLIN® (anti-CD8 monoclonal antibody), TNX-355 (CD4 antibody), AGT-1, PRO-140 (CCR5 antibody), and Anti-CTLA-4 Mab. A HIV vaccine is exemplified by, but not limited to, ALVAC® (live canarypox vector vaccine), AIDSVAX® (gp120 vaccine), REMUNE® (beta-propiolactone inactive HIV-1 vaccine), HIV gp41 vaccine, HIV gp120 vaccine, HIV gp140 vaccine, HIV gp160 vaccine, HIV p17 vaccine, HIV p24 vaccine, HIV p55 vaccine, AlphaVax Vector System, canarypox gp160 vaccine, AntiTat, MVA-F6 Nef vaccine, HIV rev vaccine, C4-V3 peptide, p2249f, VIR-201, HGP-30W, TBC-3B, PARTICLE-3B, and Antiferon (interferon-α vaccine). An interferon or interferon agonist is exemplified by, but not limited to, SUMIFERON® (interferon alpha), MULTIFERON® (interferon alpha), interferon-τ, Reticulose, Human leukocyte interferon alpha. A CCR5 antagonist is exemplified by, but not limited to, SCH-351125. An agent acting on HIV p24 is exemplified by, but not limited to, GPG-NH2 (glycyl-prolyl-glycinamide), a HIV fusion inhibitor is exemplified by, but not limited to, FP-21399 (1,4-bis[3-[(2,4-dichlorophenyl)carbonylamino]-2-oxo-5,8-di sodium sulfonyl]naphthyl-2,5-dimethoxyphenyl-1,4-dihydrazone), T-1249, Synthetic Polymeric Construction No 3, pentafuside, FP-21399, PRO-542, and Enfuvirtide. An IL-2 agonist or antagonist is exemplified by, but not limited to, interleukin-2, IMMUNACE® (advanced micronutrients), PROLEUKIN® (aldesleukin), MULTIKINE® (leukocyte interleukin), ONTAK (denileukin diftitox)®, a TNF-α antagonist is exemplified by, but not limited to, THALOMID® (thalidomide), REMICADE® (infliximab), and curdlan sulfate. A α-glucosidase inhibitor may be BUCAST® (butanoyl derivative of castanospermine). A purine nucleoside phosphorylase inhibitor is exemplified by, but not limited to, peldesine (2-amino-4-oxo-3H,5H-7-[(3-pyridyl)methyl]pyrrolo[3,2-d]pyrimidine), an apoptosis agonist or inhibitor is exemplified by, but not limited to, ARKIN Z® (vesarinone), PANAVIR® (high-molecular weight plant polysaccharides), and Coenzyme Q10 (2-deca(3-methyl-2-butenyl ene)-5,6-di m ethoxy-3-methyl-p-benzoquinone), a cholinesterase inhibitor is exemplified by, but not limited to, COGNEX® (tacrine), and an immunomodulator is exemplified by, but not limited to, IIVIMUNOX® (glucan), PROKINE® (sargramostim), Met-enkephalin (6-de-L-arginine-7-de-L-arginine-8-de-L-valinamide-adrenorphin), WF-10 (10-fold dilute tetrachlorodecaoxide solution), Perthon, PRO-542, SCH-D, UK-427857, AMD-070, and AK-602.

In addition, NEUROTROPIN® (non-protein extract of cutaneous tissue inoculated with vaccina virus), LIDAKOL® (n-docosanol 10%), ANCER 20® (Z-100), AMPLIGEN® (rintalolimod), ANTICORT® (procaine), INACTIVIN® (16alpha-Bromoepiandrosterone), PRO-2000, Rev M10 gene, HIV specific cytotoxic T cell (CTL immunotherapy, ACTG protocol 080 therapy, CD4-ζ gene therapy), SCA binding protein, RBC-CD4 complex, Motexafin gadolinium, GEM-92, CNI-1493, (±)-FTC, Ushercell, D2S, BUFFERGEL® (spermicide and microbicide gel), VIVAGEL® (SPL7013 gel), Glyminox vaginal gel, sodium lauryl sulfate, 2F5, 2F5/2G12, VRX-496, Ad5gag2, BG-777, IGIV-C, and BILR-255 are also examples of anti-HIV drugs.

The nanodrug may be conjugated to a targeting antibody, such as for delivery to the brain, lymph nodes, or gut-associated lymphoid tissue. Brain specific antibodies may be neuron specific or microglia specific, such as Tmem19, Siglec-H, anti-EGFR, anti-GP2 monoclonal antibody, or anti-IGFBP7 sdAb.

In some embodiments, the nanodrugs may comprise additional modifications. For example, the nanodrugs may be PEGylated.

“PEG”, “polyethylene glycol”, and “poly(ethylene glycol)” are used interchangeably herein to refer to a compound comprising the repeating unit —[O—CH₂—CH₂]ₙ—. For example, the PEG may comprise the structure CH₃—[O—CH₂—CH₂]ₙ— (mPEG) or H—[O—CH₂—CH₂]ₙ—. Polyethylene glycol is an example of a PEG and refers to a compound with the structure H—[O—CH₂—CH₂]ₙ— OH. As would be recognized by one of skill in the art, a wide variety of sizes of PEG may be used to pegylate an antibody, such as colistin. For example, in some embodiments, n=2-4000, 2-3000, 2-2000, 50-4000, 50-3000, 50-2000, 100-2000, 100-750, or 250-1000. In some embodiments, the PEG has a molecular weight of 100-10000, 2000-20000, or 2000-10000 daltons. In some embodiments, the PEG has a molecular weight of 1500-7500, 4000-6000, or about 5000 daltons. In some embodiments, the PEG is PEG 5k. In some embodiments, the PEG has the formula CH₃— PEG— OC(O)CH₂ CH₂C(O)OH or CH₃—[O—CH₂— CH₂]ₙ—OC(O)CH₂CH₂C(O)OH.

Modified PEG moieties are known and may comprise the formula H— [O— CH₂— CH₂]ₙ—. The n in the formula of the modified PEG have a range as defined above. The modified PEG may have the structure H—[O—CH₂—CH₂]ₙ— (leaving group), wherein the leaving group is defined below. For example, the leaving group may be, e.g., — OH (e.g., as present in —OC(O)CH₂CH₂C(O)OH or other esters), — OMs, —OTf, —OMe, or —OTs. After pegylation, the PEG moiety may be covalently bound to the aminoglycoside moiety via an amide, etc. bond. Methods for PEGylating an antibody are described in US20170143842; incorporated herein by reference in its entirety.

A “leaving group” when used as described above is a functional group which converts the hydroxyl group into a better leaving group. This functional group makes the hydroxyl group a better leaving group by stabilizing the charge on the oxygen when the atom bears a negative charge. This functional group makes the hydroxyl group more susceptible to a nucleophilic attack and displacement by nucleophilic groups.

PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also enhance biofilm penetration, mucus penetration, and provide water solubility to hydrophobic drugs and proteins.

The first step of the PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and NHS esters.

The most common modification agents, or linkers, are based on methoxy PEG (mPEG) molecules. Their activity depends on adding a protein-modifying group to the alcohol end. In some instances, polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEG is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally, the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five key parameters. The “design of experiments” approach to optimization of PEGylation conditions can be very useful.

For thiol-specific PEGylation reactions, parameters to consider include: drug concentration, PEG-to-drug ratio (on a molar basis), temperature, pH, reaction time, and in some instances, the exclusion of oxygen. (Oxygen can contribute to intermolecular disulfide formation by the drug, which will reduce the yield of the PEGylated product.) The same factors should be considered (with the exception of oxygen) for amine-specific modification except that pH may be even more critical, particularly when targeting the N-terminal amino group.

B. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged. In certain embodiments, the liposomes are neutral in charge.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a polypeptide or nucleic acids may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid (Avanti Polar Lipids, Alabaster, AL), such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

In other alternative methods, liposomes can be prepared in accordance with other known laboratory procedures (e.g., see Bangham et al., 1965; Gregoriadis, 1979; Deamer and Uster, 1983; Szoka and Papahadjopoulos, 1978, each incorporated herein by reference in relevant part). Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer. A process of making liposomes is also described in WO04/002453A1. Neutral lipids can be incorporated into cationic liposomes (e.g., Farhood et al., 1995). Various neutral liposomes which may be used in certain embodiments are disclosed in U.S. Pat. No. 5,855,911, which is incorporated herein by reference. These methods differ in their respective abilities to entrap aqueous material and their respective aqueous space-to-lipid ratios.

The size of a liposome varies depending on the method of synthesis. Liposomes in the present embodiments can be a variety of sizes. In certain embodiments, the liposomes are small, e.g., less than about 500 nm, less than about 400 nm, less than about 100 nm, about 90 nm, about 80 nm, about 70 nm, about 60 nm, or less than about 50 nm in external diameter. For example, in general, prior to the incorporation of nucleic acid, a DOTAP:cholesterol liposome for use according to the present embodiments comprises a size of about 50 to 500 nm. Such liposome formulations may also be defined by particle charge (zeta potential) and/or optical density (OD). For instance, a DOTAP:cholesterol liposome formulation will typically comprise an OD₄₀₀ of less than 0.45 prior to nucleic acid incorporation. Likewise, the overall charge of such particles in solution can be defined by a zeta potential of about 50-80 mV.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), di stearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), di stearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

### III. METHODS OF TREATMENT

In some embodiments, the present disclosure provides methods of using the nanodrugs provided herein for the delivery of a therapeutic agent, such as an anti-retroviral therapy, to a cell, such as a neuron or macrophage. In another embodiment, there is provided a method of treating a subject with a disease or disorder comprising administering an effective amount of the nanodrugs of the present disclosure.

The subject may be a human, a mouse, a rat, a rabbit, a dog, a cat, a cow, a horse, a pig, a goat, a sheep, a primate, or an avian species. In particular embodiments, the subject is a human. For example, the human may be a subject with a disease. The disease may be any disease that afflicts a subject, such as a viral disease, an inflammatory disease, a hyperproliferative disease, an infectious disease, or a degenerative disease. In some aspects, the disease is Alzheimer's disease, Parkinson's, or other neurodegenerative disorders. In certain aspects, the disease is COVID-19.

A. Pharmaceutical Compositions

Certain of the methods set forth herein pertain to methods involving the administration of a pharmaceutically effective amount of a composition comprising nanodrugs of the present disclosure.

1. Compositions

The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations preferably meet sterility, pyrogenicity, general safety and purity standards as required by FDA Office of Biologics standards.

The compositions comprising nanodrugs may be extensively dialyzed to remove undesired small molecular weight molecules and/or lyophilized for more ready formulation into a desired vehicle, where appropriate. The active compounds will then generally be formulated for administration by any known route, such as parenteral administration. Methods of administration are discussed in greater detail below.

The present disclosure contemplates methods using compositions that are sterile solutions for intravascular injection or for application by any other route as discussed in greater detail below. A person of ordinary skill in the art would be familiar with techniques for generating sterile solutions for injection or application by any other route. Sterile injectable solutions are prepared by incorporating the active compounds in the required amount in the appropriate solvent with various of the other ingredients familiar to a person of skill in the art.

The formulation of the composition may vary depending upon the route of administration. For parenteral administration in an aqueous solution, for example, the solution should be suitably buffered and the liquid diluent first rendered isotonic with sufficient saline or glucose. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, formulations for administration via an implantable drug delivery device, and any other form. One may also use nasal solutions or sprays, aerosols or inhalants in the present disclosure.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

In certain embodiments, pharmaceutical composition includes at least about 0.1% by weight of the active agent. The composition may include, for example, about 0.01%. In other embodiments, the pharmaceutical composition includes about 2% to about 75% of the weight of the composition, or between about 25% to about 60% by weight of the composition, for example, and any range derivable therein.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition may be stable under the conditions of manufacture and storage, and preserved against the contaminating action of microorganisms, such as bacteria and fungi.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, one may use nasal solutions or sprays, aerosols or inhalants in the present disclosure. Nasal solutions may be aqueous solutions designed to be administered to the nasal passages in drops or sprays.

Sterile injectable solutions are prepared by incorporating the nanodrugs in the required amount in the appropriate solvent with various of the other ingredients enumerated above, as required, followed by sterilization.

2. Routes of Administration

Upon formulation, nanodrugs will be administered in a manner compatible with the dosage formulation and in such amount as is therapeutically effective.

The nanodrugs can be administered to the subject using any method known to those of ordinary skill in the art. For example, a pharmaceutically effective amount of a composition comprising nanodrugs may be administered intravenously, intracerebrally, intracranially, intrathecally, into the substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990). In particular embodiments, the composition is administered to a subject using a drug delivery device.

3. Dosage

A pharmaceutically effective amount of the nanodrugs is determined based on the intended goal, for example inhibition of cell death. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

For example, a dose of the therapeutic agent may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, the method may provide a continuous supply of a pharmaceutical composition to the patient. This could be accomplished by catheterization, followed by continuous administration of the therapeutic agent. The administration could be intra-operative or post-operative.

B. Combination Treatments

Certain embodiments of the present disclosure provide for the administration or application of one or more secondary forms of therapies for the treatment or prevention of a disease. If the secondary therapy is a pharmacological agent, it may be administered prior to, concurrently, or following administration of the nanoparticles.

The interval between the administration of the nanodrugs and the secondary therapy may be any interval as determined by those of ordinary skill in the art. For example, the interval may be minutes to weeks. In embodiments where the agents are separately administered, one would generally ensure that a long period of time did not expire between the time of each delivery, such that each therapeutic agent would still be able to exert an advantageously combined effect on the subject. For example, the interval between therapeutic agents may be about 12 h to about 24 h of each other and, more preferably, within about 6 hours to about 12 h of each other. In some situations the time period for treatment may be extended, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the nanodrugs.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1— Development and Characterization of Targeted Nanodiscs

Studies were performed to characterize aspects of the nanodisc in order to determine its viability in vivo. In terms of structural characterization, the particle size, zeta potential (surface charge), charge, and the dissolution of the nanodisc were determined by small angle X-ray, neutron scattering (SAXS or SANS), or dynamic light scattering. For the biological characterization, the dose dependent efficacy of the nanodisc formulation and toxicity of the nanodisc were determined. In addition, a Reactive Oxygen Species (ROS) Assay was performed in order to determine the production of ROS in the system.

A small dosage of anti-HIV drug that can reach therapeutically beneficial levels of treatment was encapsulated in the nanodisc formulation and inserted into an in vitro cell culture model of the BBB. From there, the activity of the nanodisc was monitored to ensure the delivery of the nanodisc into the BBB past the HBMVEC. Once the nanodisc was delivered into the in vitro model of the BBB, the slow release of the drug was monitored inside the model and the half-life of the model was measured as well as effectiveness of the slow release of the drug into the brain to determine the extended period of time the nanodisc keeps the drug viable.

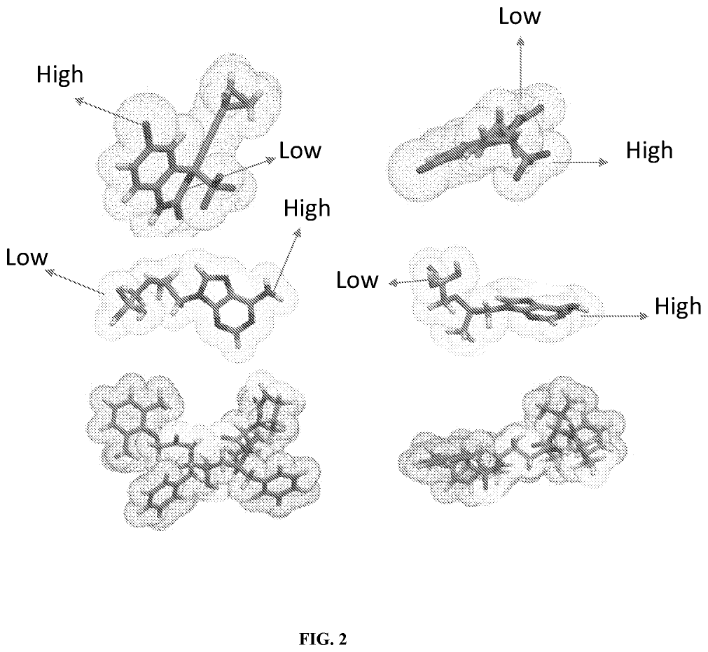

The molecular electrostatic potentials (MEP) and MLSP of TFV were calculated using the VEGA-ZZ software. MEP illustrates the 3D charge distributions within molecules, and MLSP demonstrates the lipophilicity potential in the different regions of the molecules. MEP and MSLP provide insights into the molecular structure, lipophilicity, and surface charge of the drugs and characterize TFV interactions with the phospholipids and their entrapment within the NDs. Furthermore, both MLSP and MEP are valuable tools for understanding the self-assembly process of drug-loaded NDs and encapsulation. In FIG. 2, hydrophilic and hydrophobic moieties of each drug were traced using MLSP of the molecule, which shows sites of high and low LP for Efavirenz and Tenofovir, respectively. These lipophilicity analyses explained why some molecules entrap easily into the bilayer and some do not. As shown in FIG. 2, TFV is a highly lipophobic molecule, limiting its incorporation within the bicellar core. TFV prefers to locate in the external terminals of the phospholipids due to their higher hydrophilicity.

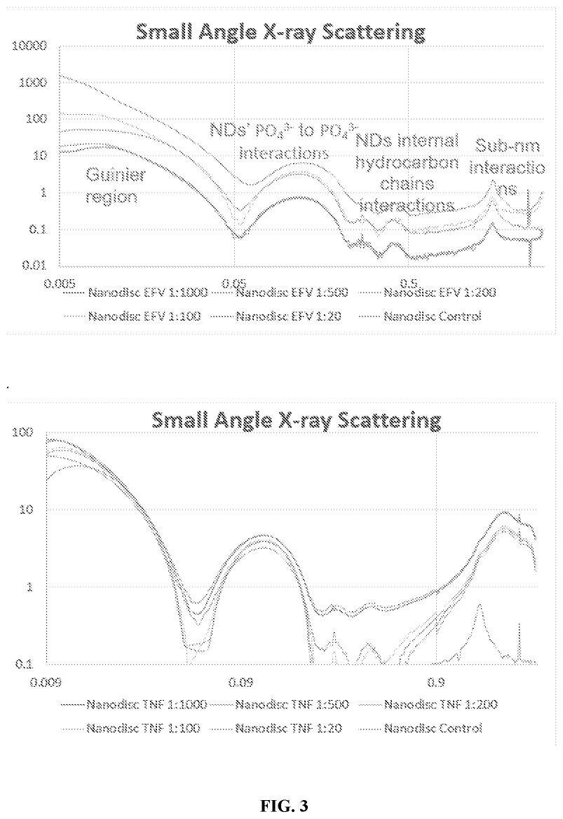

SAXS was used to provide the detailed discoidal core-shell architecture of the designed nanoparticles (FIG. 3). This technique is very sensitive to the electron density distribution in the structure of nanoassemblies averaged in time. The lipid bilayers are a lipophilic core (hydrocarbon chains) sandwiched by two shells (hydrophilic phosphatidylcholine head groups). Since the phosphate group has the highest electron density in the system—higher than those of hydrocarbon tails and water, the electron density profile across the bilayer (i.e., water-head group shell-hydrocarbon core-head group shell-water) can be approximated by a “square well”. As a result, the SAXS pattern led to a broad peak corresponding to the correlation length of head group-head group distance as shown. Moreover, the slope at low q region inferred possible morphology of the lipid aggregates.

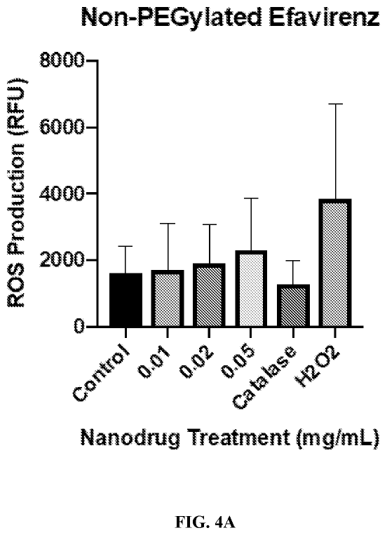

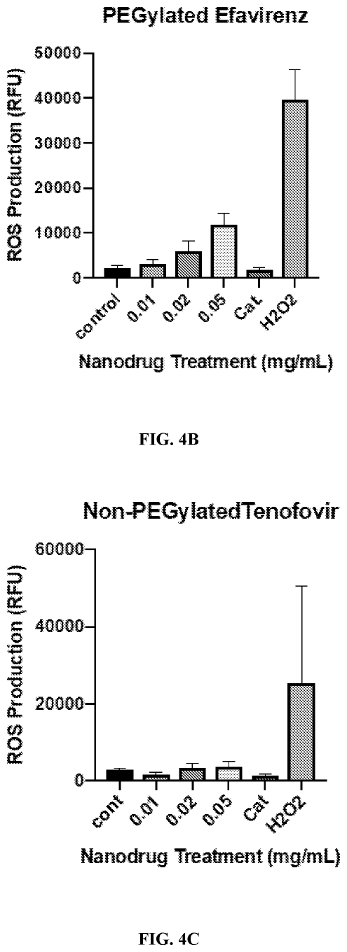

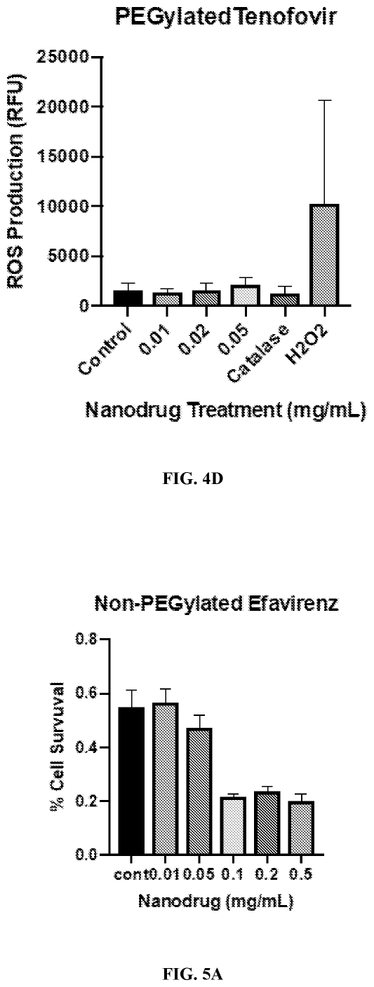

The ROS assay was performed at different concentrations of the nanodisc formulation for non-PEGulated Efavirenz (FIG. 4A), PEGylated Efavirenz (FIG. 4B), non-PEGylated Tenofovir (FIG. 4C), and PEGylated Tenofovir (FIG. 4D). The ROS production was measured in nanodisc formulation treated cells and compared with untreated control. No significant change in ROS production was observed with increasing concentration of nanodisc formulation.

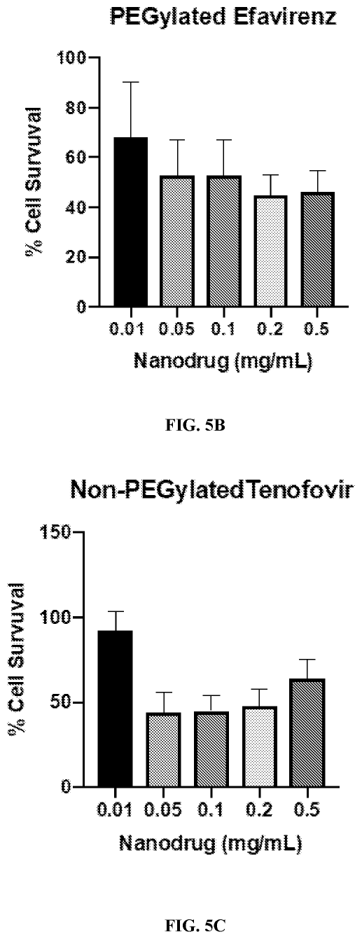

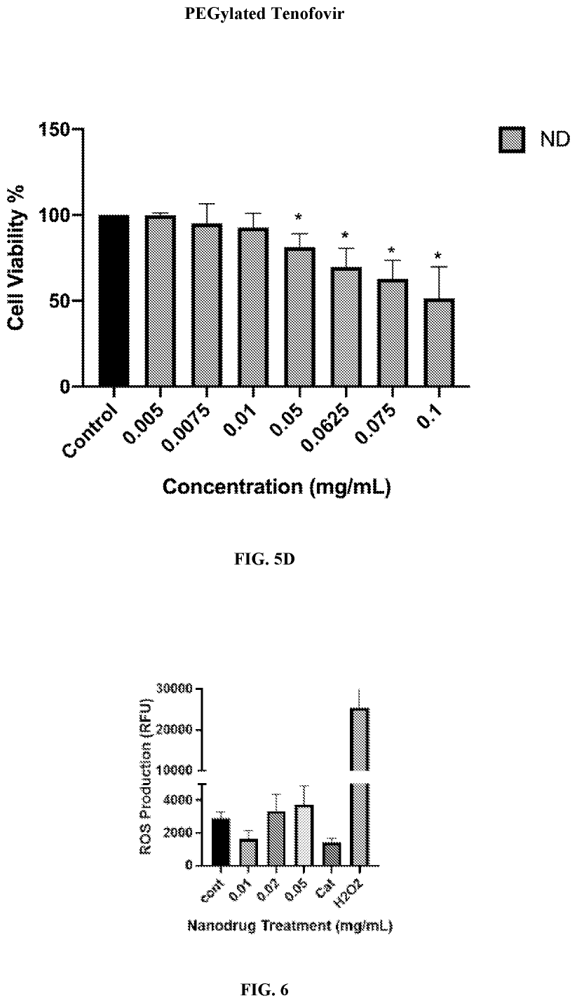

SK-N-MC cells were treated with 0.1 mg/mL nanodisc formulation for 24 hours including non-PEGylated Efavirenz (FIG. 5A), PEGylated Efavirenz (FIG. 5B), non-PEGylated Tenofovir (FIG. 5C), and PEGylated Tenofovir (FIG. 5D). After incubation, MTS assays were preformed and optical density was measured at 490 nm. The difference in the concentrations was observed to have no significant effect on cell survival.

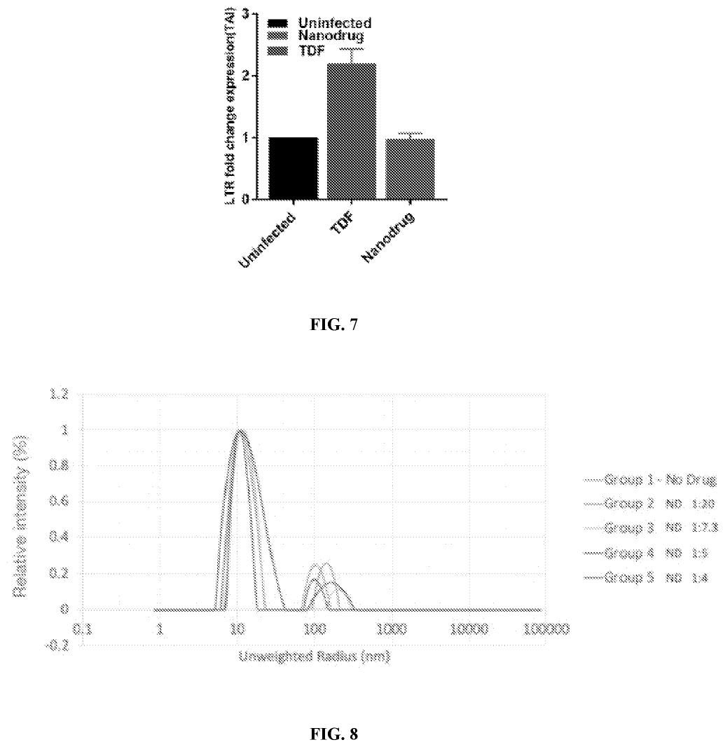

It was observed that the nanodrug with the anti-HIV drug tenofovir (TDF) did not have any toxicity to the neuronal cells SK-N-MC up to 0.01 mg/ml. It also did not induce any Reactive Oxygen Species (ROS) production with the increasing concentrations of nanodrug (FIG. 6) indicating that the nanodrug was stable, biocompatible and nontoxic to the brain cells. In addition, it was also observed that unlike unformulated TDF, the nanodrug alone can significantly inhibit the HIV latent gene expression (HIV-LTR) beyond detection level in HIV-1 infected primary human macrophages after one week of incubation (FIG. 7). This data strongly supports the potency of the nanodrug in controlling the virus with the one-week treatment of a single dose. The uninfected macrophages served as negative control and the LTR gene expression in both cells were expressed in transcript accumulation index (TAI) and normalized with housekeeping gene GAPDH. Thus, the nanodrug can be therapeutically effective in sustained release of TDF with limited side effects.

The size distribution of drug-loaded nanodiscs (NDs) was also investigated using dynamic light scattering (DLS). The hydrodynamic radius (Rh) of the NDs using DLS in an aqueous solution was used to determine the pristine and drug-loaded NDs' size distribution. In the pristine NDs without any drugs, Rh was approximately 8.5 nm. The hydrodynamic radius, Rh of NDs was 10˜13 nm. After drug encapsulation and at lower drug concentrations (drug:lipid ratios of 1:20), a slight increase in size was observed which can be observed in group 2's broader peak. Larger aggregates of the ND sample were observed within the sample but are considered as the minority and are observed as 1 in 10,000 particles. The DLS results confirmed the uniformity of the nanodiscs for individual TFV-ND samples (FIG. 8).

SAXS was used to provide the detailed discoidal core-shell architecture of the designed ND (FIG. 9). This particular technique is sensitive to the electron density distribution in the structure of nano-assemblies averaged in time. The pattern of the x-axis is 1/Angstrom; thus, 33 higher x-values correlate to smaller distances. Lipid bilayers within the nanodisc are considered as the lipophilic core, represented in SAXS data as hydrocarbon chains, being sandwiched between two shells of hydrophilic phosphatidylcholine head groups outside. Since the phosphate group has the highest electron density in the system and is higher than hydrocarbon tails and water, the electron density profile across the bilayer can be approximated by a “square well.” Moreover, the slope at the low q region could also infer the lipid aggregates' possible morphology.

The ND formulation was prepared at different drug to lipid ratios (1:4 to 1:20). This drug to lipid ratio was varied based on the drug encapsulating capacity at the core of the ND. The amount of bicellar lipid significantly contributed to the drug loading capacity of the ND formulation. Therefore, formulations with the lowest (1:4) and highest (1:20) lipid concentrations were selected for further study. The difference in 1:4 and 1:20 drug-to-lipid ratios was the amount of lipid content within the ND's bicelle; however, the amount of drug concentration for both remained the same.

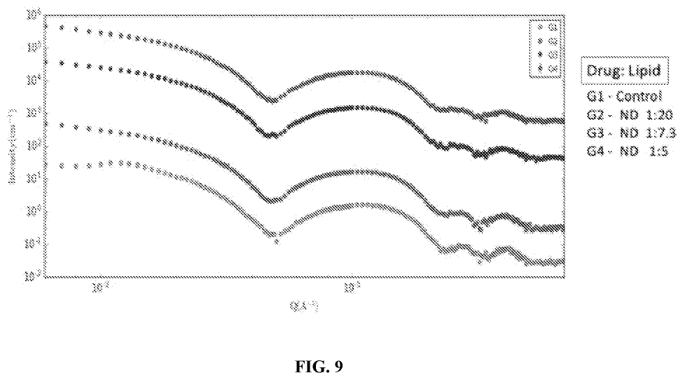

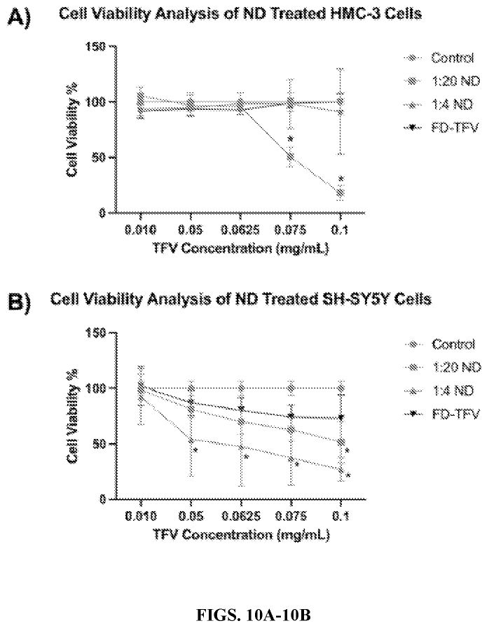

To characterize the nanodisc formulation in the biological system in in vitro conditions, it was necessary to screen the formulation for cytotoxicity and ensure that it would not induce significant cytotoxicity to microglial (HMC-3) and neuronal (SH-SY5Y) cells. Since the formulation was developed to target the brain, it was important to observe the effect of ND on these cells for any neurotoxicity or cytotoxicity. A cell viability (MTS) study was performed for both 1:20 and 1:4 NDs as per the manufacturer's instruction (G3582, Promega, Madison, WI, USA). Nanodiscs were introduced to these cells separately at varying TFV drug concentrations of 0.01-0.1 mg/mL and incubated for 72 hours. Overall, cell viability results indicated that ND was less toxic to HMC-3 cells than SH-SY5Y cells. The 1:20 ND formulation was found to be significantly toxic at concentrations above 0.075 mg/mL for HMC-3 cells (FIG. 10). For SH-SY5Y cells, 1:20 ND was found to be only statistically significant at 0.1 mg/mL but was observed to have a decrease cell viability starting at 0.05 mg/mL. The 1:4 ND formulation was significantly toxic at concentrations above 0.05 mg/mL for SH-SY5Y cells. Free drug-TFV or unformulated TFV (FD-TFV) was not considered to be significantly toxic when compared to the control.

A separate setup to evaluate the effect of empty NDs was done as well, where HMC-3 and SH-SY5Y cells were treated with varying lipid concentrations of the bicellar ND which associate with the TFV concentrations used prior (Table 3). Results showed that lipid concentrations above 0.08% and 0.05% were considered significantly cytotoxic for HMC-3 cells and SH-SY5Y cells, respectively (FIG. 11). This indicated that at higher drug concentrations associated with higher lipid concentrations, there is an observed decrease in cell viability.

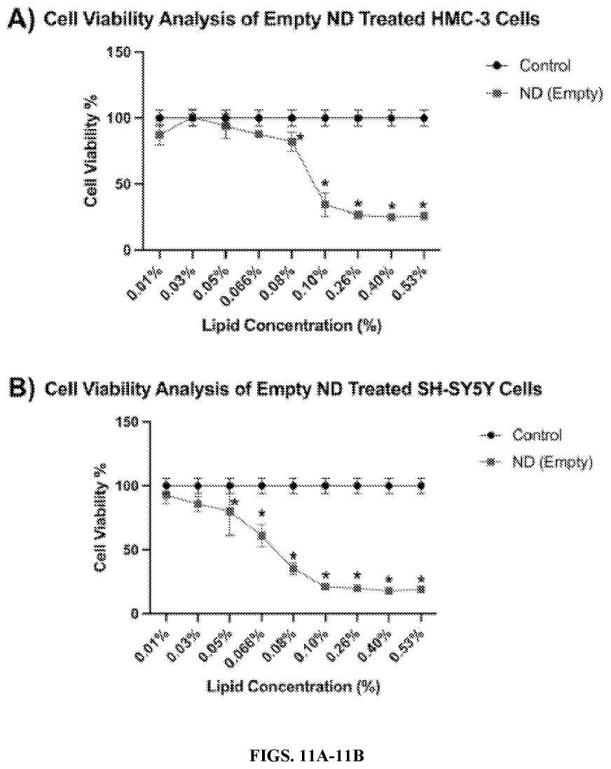

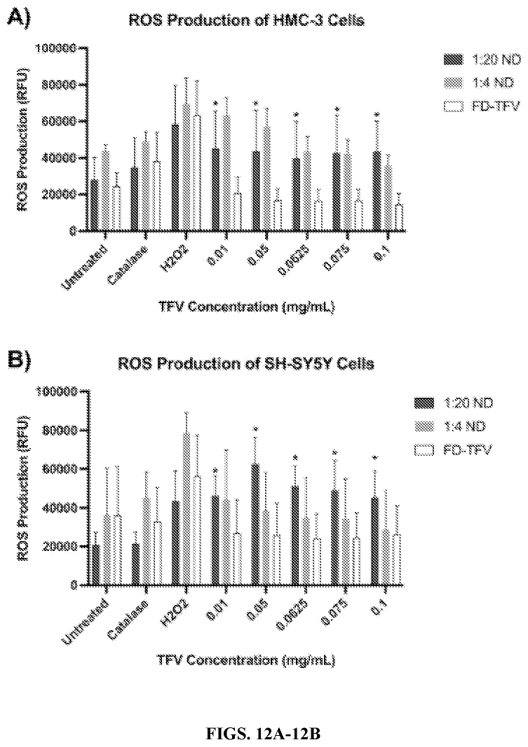

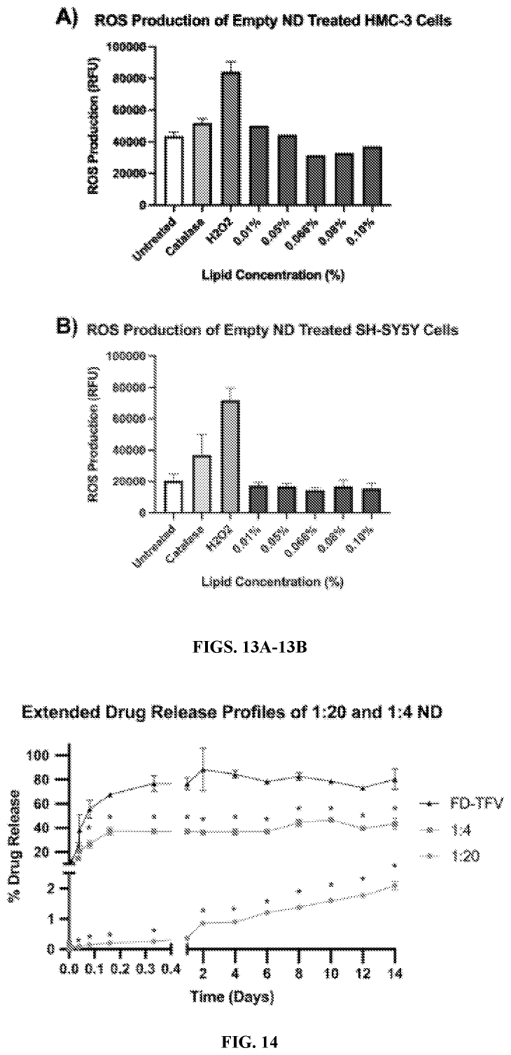

ROS production by ND on HMC-3 and SH-SY5Y cells: While nanomaterials possess unique properties that have increased their use, understanding their interactions within biological systems is important to note. As a first approach to predict the inflammatory response on neuronal cells, the ROS production of HMC-3 and SH-SY5Y cells was evaluated. HMC-3 (FIG. 12A) and SH-SY5Y cells (FIG. 12B) were treated with the different drug-to-lipid ratios of 1:20 and 1:4 at varying TFV drug concentrations (0.01-0.1 mg/mL). A negative control antioxidant (catalase) and positive control (H₂O₂) were used on non-treated cells. A simultaneous setup was also performed for empty NDs at varying lipid concentrations. Since a significant drop and then a plateau in cell viability was seen for lipid concentration above 0.1%, ROS assay was only done for lipid concentrations of 0.01%-0.1% to observe ROS production (FIG. 12).

Both HMC-3 cells and SH-SY5Y cells undergoing the treatment of 1:20 ND were shown to significantly increase ROS production at all of the tested concentrations (0.01-0.1 mg/mL) compared to the control (FIG. 12). SH-SY5Y cells were undergoing the treatment of 1:4 ND did not show a significant increase in ROS production at tested concentrations up to 0.1 mg/mL for both cell lines.

The FD-TFV's effect on ROS production of HMC-3 and SH-SY5Y cells was also analyzed. For both cell lines, the FD-TFV did not induce a significant increase in ROS production, indicating that the FD-TFV does not actively cause inflammatory properties at the tested concentrations. Overall, these results indicated that ROS production in 1:4 ND treated cells was closer to untreated cells.

In vitro sustained drug release assay: The nanodisc formulation's extended drug release profile was determined in phosphate buffer saline (PBS) using an equilibrium dialysis system. The released TFV from the dialysis bag into the outside environment was sampled at different time points up to 14 days and was measured by LC-MS/MS. The dialysis bag used in these experiments was selected for its pore size of 6-8 kDa MWCO. The nanodisc typically had an average molecular weight of 3.7 kDa without drug, so the selection of 6-8 kDa MWCO was selected so that drug transport would not be a limiting factor. Simultaneously, a separate set up with the same concentration and volume of FD-TFV was used as a control. Results were expressed as the percentage of total TFV released into the system compared to the initial concentration. It was observed that 63% of FD-TFV was release within 4 hours. Compared to FD-TFV, the 1:4 ND showed 35% drug release within 4 hours (FIG. 14). Compared to the FD-TFV, the 1:20 ND showed 0.18% drug release within 4 hours and showed a significant, sustained drug release of TFV, in vitro condition. (FIG. 14).

Overall, when comparing both 1:20 ND, 1:4 ND, the FD-TFV had a significantly faster TFV release, although it is considered delayed in terms of free drug. However, between 1:20 ND and 1:4 ND, 1:20 was shown to have a significantly extended release than 1:4 ND (FIG. 14).

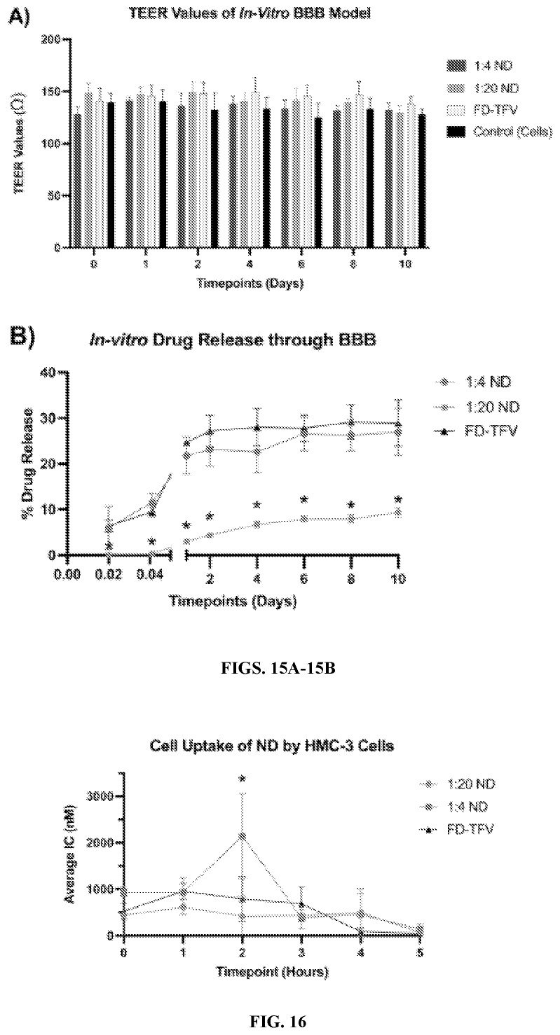

Drug release in vitro environment through the BBB: The ND was further investigated for its drug delivery property crossing the BBB. An in vitro BBB model was set up to mimic the biological barrier encountered in drug delivery towards HIV-1 residing in the brain and understand the drug release kinetics of the ND in the BBB environment. In this experiment, an in vitro BBB model was set up. The BBB model's integrity was characterized by measuring the TEER values before and after the ND exposure. The range of TEER values was from 130 to 150Ω at the beginning and the end of the experiments (FIG. 15A). The range of TEER values was similar to all three groups including the control, ND and FD-TFV groups. The TEER values indicated the consistent integrity of BBB throughout the experiment, confirming the contribution of BBB in transporting the ND formulation from the apical to basolateral side of BBB. The percentage of drug release of TFV from 1:4 ND formulation was compared with the FD-TFV and the pattern indicated that the 1:4 ND has a sustained release up to four days, which was not significantly different from FD-TFV (FIG. 15B).

Based on the results of this in vitro BBB model, the 1:4 ND was observed to have similar drug release characteristics when compared to FD-TFV. In contrast, the 1:20 ND formulation was shown to have extended-release properties and was found to be significantly different from that of the FD-TFV. These results are similar to those seen in the dissolution study (FIG. 14).

Cell uptake and characterization of extended-release from nanodisc: To evaluate the intracellular uptake of TFV in microglial cells an in vitro cellular uptake study was performed. The cellular uptake of the two formulations (1:20 ND and 1:4 ND) and FD-TFV as a control were evaluated simultaneously and the uptake and release of TFV was monitored over 24 hours. The results showed different uptake within the formulations. Maximum uptake was observed at 1 hour for 1:20 ND, 2 hours for 1:4 ND, and 2 hours for FD before trending down (FIG. 16). The concentrations measured within the samples were found to be below or around the limit of quantification; thus, only a small amount of TFV can cross the cell membrane. This small window of cellular uptake may indicate that there is a certain capacity that microglia cells can uptake TFV. Observed results from this cellular uptake study indicated that 1:4 ND is observed to accumulate tenofovir within the cell better than 1:20 ND and FD-TFV formulations. However, the short windows of drug uptake by the microglial cells could indicate that the size and surface charge of the ND could be a contributing factor.

In vivo maximum tolerated dose study and pharmacokinetic analysis of ND: During the MTD study, male and female BALB/c mice were treated with a single iv dose administration at 0, 2, 10, 15, or 20 mg/kg. Mice were observed at pre-determined timepoints, appeared normal throughout the phase, and tolerated the ND's varying concentrations well. The highest MTD dose level of TFV was selected at 20 mg/kg to continue with the PK study.

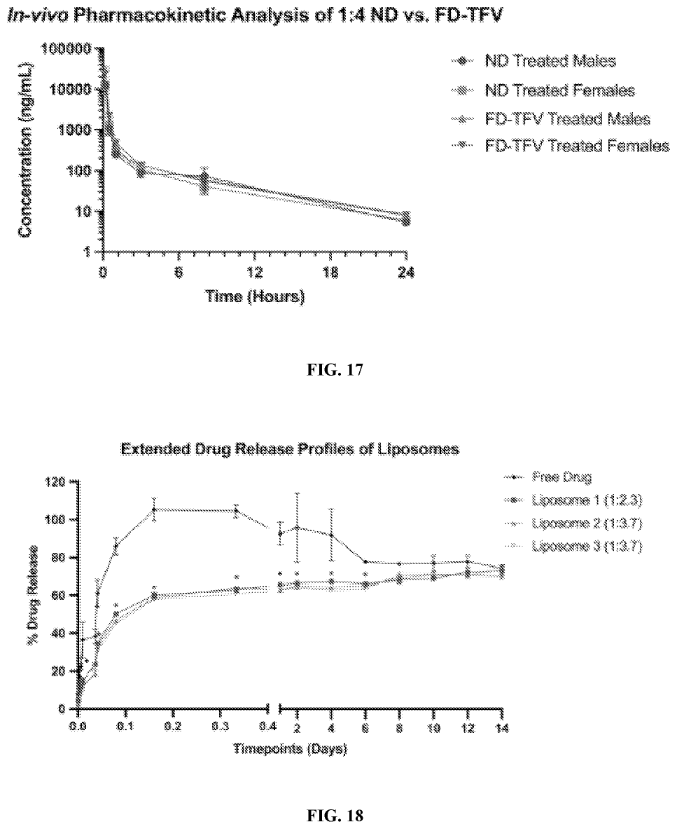

During the PK study, male and female BALB/c mice were administered a single dose iv administration at 20 mg/kg TFV in ND formulation (Group 6) and unformulated (FD-TFV) tenofovir in sterile saline formulation (Group 7). PK data analysis was performed using the plasma concentrations of TFV in the mice (FIG. 17). Plasma samples were collected for up to 48 hrs and 72 hours. The plasma concentration of ND formulation were 44,300 ng/ml (males) and 34,400 ng/ml (females), and for FD-TFV (Group 7) were 46,000 ng/ml (males) and 79,300 ng/ml (females) (Table 4). Statistical analysis indicated significance amongst FD-TFV treated females, this may due to individual animal variation as one mouse had plasma concentrations about 2-fold higher at the first time point than the other females within the group. Exposure based on area under the plasma concentration curve to the last time point (AUClast) values for the lipid formulation was 8,710±195 hr·ng/ml and 7,590±686 hr·ng/ml for males and females, respectively. In the mice receiving TFV in a sterile saline formulation, the area under the plasma concentration curve to the last time point (AUClast) values was higher, 9,870±697 hr·ng/ml (males) and 15,200±1,680 hr·ng/ml (females). The AUCinf values were higher in unformulated TFV (Group 7) than ND formulation (Group 8).

TABLE 1
Effect of ND and FD-TFV on PK in male and female
BALB/c mice after IV administration, 20 mg/kg.

| | | t_{1/2} | C₀ | AUCₗₐₛₜ | Cl (mL/ | Vz |
|---|---|---|---|---|---|---|
| Group | Sex | (hr) | (ng/mL) | (hr · ng/ml) | hr/kg) | (mL/kg) |
| ND | M | 4.9 | 44,300 | 8,710 | 8,750 | 16,300 |
| ND | F | 5.2 | 34,400 | 7,590 | 7,630 | 19,800 |
| FD-TFV | M | 5.3 | 46,000 | 9,870 | 9,930 | 15,500 |
| FD-TFV | F | 5.3 | 79,300 | 15,200 | 15,300 | 9,930 |

TABLE 2
Drug-to-lipid lipid concentration at associated drug concentrations.

| | |
|---|---|
| TFV Concentration | Lipid Concentration (%) |

| (mg/mL) | 1:4 ND | 1:20 ND |
|---|---|---|
| 0.01 | 0.01 | 0.05 |
| 0.05 | 0.05 | 0.26 |
| 0.0625 | 0.066 | 0.33 |
| 0.075 | 0.08 | 0.4 |
| 0.1 | 0.1 | 0.53 |

The clearance (Cl) values for Group 6 were higher than the Cl values observed for Group 7. The volume of distribution (Vz) was high for all groups with values greater than 10 mL/kg; this result is consistent with a drug accumulating intracellularly, such as TFV. The terminal t_{1/2} was 4.9 hr and 5.2 hr for Group 6 males and females, respectively. For Group 7, the terminal t_{1/2} was 5.3 hr for both sexes.

Overall, some differences in the PK of TFV when administered encapsulated in ND compared to FD-TFV. In male mice, the PK parameters varied slightly between the two formulations, with AUC values about 10% higher for FD-TFV. Administration of TFV as FD-TFV in female mice resulted in plasma exposure that was 2 fold higher base on mean value of CO, AUClast and AUCinf while Cl and Vz values were lower.

Thus, the study established that nanodiscs can provide drug stability and sustained drug release properties to the TFV formulation.

In vitro sustained drug release assay of liposomal structure: Further, liposomes were formed and the pharmacokinetics were characterized. Liposomes are similar to the ND formulation as they are made up of the same molecular weight, chemical composition, lipid concentration, appearance and pH as the ND (Table 3). Liposomes can encapsulate TFV within their structure due to the aqueous core which has a slow release of the drug.

TABLE 3
Comparison Table of ND vs. Liposomal Structure.

| Test | Nanodisc | Liposome |
|---|---|---|
| Molecular Weight | 760 | 760 |
| Size (Hydrodynamic | ~7.5-13 nm | ~400 nm |
| Radius) | | |
| Drug-to-Lipid Ratios | 1:4 | 1:2.3 |
| | 1:20 | 1:3.7 |
| Shape | Discoidal | Spherical |
| Active | DPPC, DSPE-PEG, | DPPC, DPPG |
| Pharmaceutical | DHPC, DPPG | |
| Ingredient (API) | | |
| pH | 6.4 | 6.4 |
| Lipid Concentration | 5% | 5% |
| Appearances | Clear, colorless | Clear, colorless |
| Stability | 1 week at 4° C. | 1 week at 4° C. |
| Storage Conditions | At room temperature | At room temperature |
| | (25° C.) | (25° C.) |

In order to study the liposome formulation's extended drug release kinetics, a drug release study was done in PBS using an equilibrium dialysis system. Three versions of the liposome-TFV were synthesized at varying drug-to-lipid ratios: liposome 1 (1:2.3), liposome 2 (1:3.7) and liposome 3 (1:3.7) (Table 3). Liposomes 2 and 3 had the same drug-to-lipid ratios but different lipid charge densities of 2% and 5%, respectively. Liposome 1 has a lipid charge density of 5%. The released TFV from the dialysis bag into the outside environment was sampled at different time points up to 14 days and was measured by a NanoDrop Microvolume Spectrophotometer. Simultaneously, a separate setup with the same concentration and volume of FD-TFV was used as a control. Results were expressed as the cumulative total of TFV released into the system compared to the initial concentration (FIG. 18). It was observed that 100% of the FD-TFV was released within 4 hours; whereas Liposomes 1, 2, and 3 showed a 56-57% drug release of TFV within 4 hours. This indicated the liposomal formulations had a sustained drug release up to 4 days when compared to FD-TFV. FD-TFV was observed to have a significantly faster drug release than the liposomes; however, it was observed to trend downward for the later timepoints of the experiment. This downward trend may be caused by byproducts from the FD-TFV in the external environment. Overall, results from this in vitro drug release study indicated that the liposome formulations have a more sustained drug release compared to FD-TFV.

### Example 2—Materials and Methods

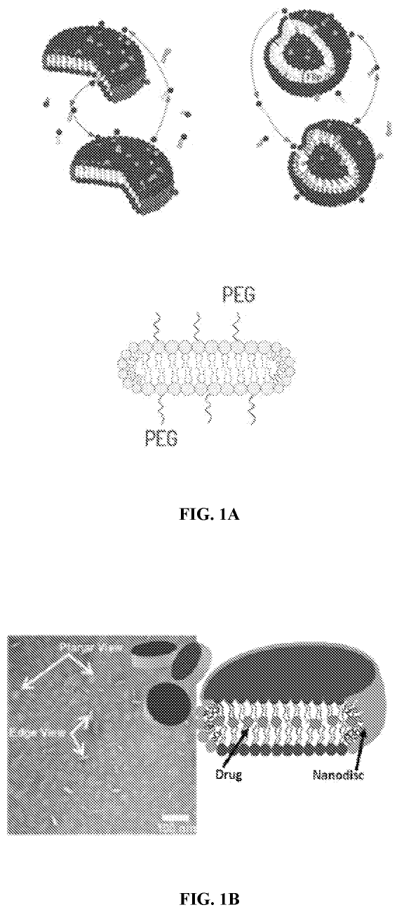

Nanodisc Sample Preparation: In FIG. 5, the lipids were prepared using a fixed molar ratio of ([DMPC]/[DMPG]/[DHPC]=50/0.5/15) and the drug-to-lipid molar ratios can vary from 1:10 to 1:1000. All chemicals were first dissolved in chloroform and dried in vacuum after complete homogenization. Then, the solid sample was re-dispersed in H2O or D2O or buffer (depending on the use of structural characterization or application for biological response) at an initial total lipid concentration of 20 wt. %, as described previously, and then stored at a temperature of 4° C. (Nieh et al., 2003). Refer to FIG. 1B for the diagram of the lipid structure (Mahabir et al., 2013).

All solvents (methanol, ethanol, chloroform, and toluene) were purchased from Sigma-Aldrich and filtered with a 0.2 μm filter before use. Zwitterionic long-chain dipalmitoylphosphatidylcholine (di-16:0, DPPC), charged long-chain dipalmitoyl phosphatidylglycerol (DPPG), zwitterionic short-chain dihexanoyl phosphatidylcholine (di-6:0, DHPC), and polyethylene glycol (PEG2000)-conjugated distearoyl phosphoethanolamine (DSPE-PEG2000) were purchased from Avanti Polar Lipids (Alabaster, AL, USA) and used without further purification. TFV, DiR dye, phosphate-buffered saline (PBS), and all pharmacological inhibitors were purchased from Sigma-Aldrich (St. Louis, MO).

NDs were prepared via self-assembly, as previously described (Liu et al., 2017). The lipids or lipids+drugs at desired ratios were homogenized in a solution of chloroform and methanol (13:7). The organic solvents were dried through a nitrogen purge at 55° C. and desiccated at room temperature overnight in a vacuum oven to remove any residual solvent. The dry lipid or lipid+drug mixtures were homogenously hydrated with filtered deionized water to 10 wt. % through temperature cycling and vortexing. The experiments were performed with the samples diluted to 1.0 or 0.1 wt. %. The drug-loaded nanodiscs were further centrifuged at 5000 rpm for 10 minutes to separate the unencapsulated drugs and large drug/lipid complexes. The experiments were performed with the samples diluted to 1.0 or 0.1 wt. %. The drug-loaded NDs were further centrifuged at 5000 rpm for 10 minutes to separate the unencapsulated drugs and large drug/lipid complexes. The lipid composition remained constant throughout the samples (DPPC:DHPC:DPPG:DSPE-PEG2000=66.6:25.1:3.8:3.8) drug-to-lipid molar ratios studied were 1:20 and 1:4. Drug-to-lipid ratios are considered theoretical and based on how much lipid and drug is being used in the whole solution, which can be calculated to know the amount of lipid and drug to add.

Liposomes were formed in a similar process as the NDs transformed into vesicle shaped through a prolonged incubation (48 hours). A disc-to-vesicle structural transition occurred as the long-chain lipid underwent the low-temperature gel (order) to high-temperature La (liquid disorder) phase. Both nanodiscs and liposomes exhibited uniform dimensions. At low lipid concentrations, the liposomes irreversibly formed and did not revert to nanodiscs even when the long-chain lipid became gel phase at a lower temperature (Nieh et al., 2009; Nieh et al., 2011; Nieh et al., 2005; all incorporated herein by reference in their entirety). These properties allowed the production of different low-polydispersity morphologies from mixtures of identical chemical compositions (Nieh et al., 2009).

Zeta Potential: The Zeta potential was within 30 minutes of sample preparation. The measurements were recorded in triplicate, and the averages of the results were used for data representation purposes.

Small- and wide-angle X-ray scattering (SAXS): SAX/WAXS was used to analyze the nanostructure of the NDs. Before measurements, samples were ultra-sonicated in a water bath for 30 min and vortexed for 10 minutes. All samples were tested at lipid concentrations of 10 mg/mL. SAXS/WAXS measurements were conducted at 16ID-LiX Beamline at National Synchrotron Light Source II, located at the Brookhaven National Laboratory (Upton, NY), using the standard flow-cell-based solution scattering setup with x-ray energy of 13.5 keV. The SAXS/WAXS intensity was expressed as a function of the scattering vector,

$$q ( q ≡ \frac{4 π}{λ} sin \frac{θ}{2} ,$$

where θ is the scattering angle) varies from 0.005 to 2.5 Å-1. Radial averaging and q-conversion of data were performed using the standard software by merging the data collected from all three detectors in the measurements. The transmission correction and background subtraction were performed to minimize the hydrogen bond's intensity from water at ˜2.0 Å-1.

For each experiment, 70 μl of the solution was transferred to Brucker Company standard quartz capillary of 1.5 mm path length, and the background of each sample was measured separately before each experiment. Both scattering and transmittance of each sample were measured separately. SAXS measurements were conducted at an NSF-MRI granted Bruker NanoSTAR instrument. X-ray was generated by a Turbo (rotating anode) X-ray source (TXS). A wavelength, λ of 1.542 Å was chosen by Cu-k_{α} using the Goble mirror. A pair of “scatterless” pinholes with diameters of 500 and 350 μm, respectively, was used for collimation. The 2-D intensity data was collected by a MikroGap VANTEC-2000 detector with a sample-to-detector distance of 108 cm to cover a scattering vector, q range

$$( q ≡ \frac{4 π}{λ} sin \frac{θ}{2} ,$$

where θ is the scattering angle) from 0.007 to 0.25 Å⁻¹ SAXS intensity is expressed as a function of q. Radial averaging and q-conversion of data were performed using the standard software.

Dynamic Light Scattering (DLS): Size and population distribution of folate and non-folate nanodiscs and vesicles were determined by ALV/CGS-8F/4 (ALV compact system, Germany) instrument which is equipped with a 632.8 nm laser beam. Both folate conjugate bicellar nanodiscs and nanovesicles were characterized. The samples were dissolved in ultrapure distilled filtered water to 0.1 wt % and vortexed before each measurement. The results were the average of 10 times measurements.

Calculation of molecular lipophilic surface potential (MLSP): MLSP describes the combined lipophilic influence of all fragments of a molecule and can be calculated at given points in space. MLSP analysis of the drug encapsulated bicelles was carried out using the Molinspiration Property Calculation Service molecular modeling package in order to study the feasibility of encapsulation of them inside the lipid bilayers of nanodiscs. The Gasteiger—Hückel charges were assigned to the atoms of TMS structure, and surfaces were generated. The color ramp for the MLSP ranges from violet/blue color representing the higher lipophilic potential (LP) and the red color representing the lower LP.

Computational molecular modeling and calculations: Molecular simulation studies were carried out to understand the interactions between the two drugs and the lipid molecules. The molecular lipophilic surface potential (MLSP) and molecular electrostatic potential (MEP) of TFV were investigated using VEGA-ZZ 3.2.0 software. MEP depicts the 3D charge distributions of the molecules. MLSP simulates the combined lipophilicity of a molecule's fragments at given points in space using the Molinspiration Property Calculation Service molecular based on the Gasteiger-Hückel charges of the atoms. The color ramp for the MLSP ranges from violet/blue (higher lipophilicity or more significant lipophilicity potential (LP) to red (lower lipophilicity or lower LP).

Zeta Potential: The Zeta potential was measured within 30 minutes of sample preparation in PBS. The measurements are recorded in triplicate, and the averages of the results are used for data representation purposes using a 90Plus Particle Size Analyzer (Brookhaven Instruments Corporation, Holtsville, NY, USA).

Microglia and Neuroblastoma Cell Culture: Human embryonic microglial clone 3 cells (HMC-3) and human neuroblastoma cells (SH-SY5Y) were purchased from the American Type Culture Collection (ATCC) Manassas, VA, USA). The transformed cell lines retain the properties of primary cells. They represent homogeneous cell populations that can be grown indefinitely and might represent a convenient system for their functions' biochemical analysis. Cells were cultured in Eagle's Minimum Essential Medium (EMEM) (ATCC®, Manassas, VA, USA) supplemented with a fetal bovine serum to a final concentration of 10% at 37° C. in a humidified, 5% CO2 atmosphere as recommended by the supplier.

Cell viability assay of ND: The nanodisc's cell viability was determined via a 3-(4,5-dimethylthiazol-2-yl)-5-(3-carboxymethoxyphenyl)-2-(4-sulfophenyl)-2H-tetrazolium (MTS) assay on HMC-3 and SH-SY5Y cells. Cells were cultured in 96-well black bottom plates at 50,000 cells per well and incubated at 37° C. in a humidified, 5% CO2 atmosphere for 24 hours to allow for 70% confluence. After 24 hours, cells were treated with various nanodisc concentrations (0.005-220.2 mg/mL) for 72 hours. The same concentration of unformulated TFV (Sigma Aldrich, Milwaukee, WI, USA) (also called free drug or FD-TFV) was also measured in similar conditions simultaneously. Untreated cells incubated with solely fresh growth medium were considered as controls. After incubation, cells were washed and incubated with 100 μl of fresh respective growth medium. Cells were incubated with 20 μl of MTS reagents (CellTiter 96® AQueous One Solution; Promega) along with the 100 μl of cell media for 1 hour at 37° C. in a humidified, 5% CO2 atmosphere. After incubation, absorbance readings at 490 nm were taken using a BioTek Synergy HTH.T. multi-mode microplate reader (BioTek, Winooski, VT, USA) every hour following for 4 hours for a total of five measurements. MTS assay was performed and optical density of culture supernatant was measured at 490 nm The net absorbance (Sample Absorbance—Absorbance of Blank) was taken as an index of cell viability of treated and untreated cells. All measurements were taken as three independently replicated experiments of give values each. Cell viability was calculated as the following equation: SampleControl×100%.

Reactive oxygen species of ND: Reactive oxygen species (ROS) productions in HMC-3 and SH-SY5Y cells following nanodisc treatment were detected using dichlorofluorescein diacetate assay (DCF-DA; Molecular Probes, Eugene, OR). Cells were cultured in 96-well black bottom plates at 100,000 cells per well and incubated at 37° C. in a humidified, 5% CO2 atmosphere for 24 hours to allow for 70% confluence. The following day, cell media was taken out from each well and replenished with 100 μl of PBS+1% FBS. Pre-assigned negative control cells were treated with antioxidant catalase, and the plate was incubated for 2 hours. After incubation, cell media was taken out and replenished with 10011M dichlorofluorescein diacetate assay (made with PBS+1% FBS) to each well and incubated for 1 hour. Following incubation, cells were treated with various concentrations (0.01-0.1 mg/mL) of the nanodisc and incubated for 2 hours. A study of FD-TFV was also measured in similar conditions simultaneously. Cells with no drug (untreated) were incubated with a growth medium and considered the control. Cells were also treated with H₂O₂ (50 μM) as triplicates positive controls. Following 2 hours, the first reading of cell ROS production was read in a BioTek Synergy HTH.T. multi-mode microplate reader (BioTek, Winooski, VT, USA) and then taken every hour for the following 18 hours (excitation 485 nm and emission 528 nm; BioTek). All measurements were taken as three independently replicated experiments of give values each.

In vitro sustained drug release assay of ND: The drug release kinetics of the nanodisc-based drug delivery was determined in PBS using equilibrium dialysis. A 50 μL solution of the 5 mg/mL nanodisc formulated TFV along with 1 mL of PBS was placed into a dialysis bag (Pur-A-Lyzer™ Maxi 6000 Dialysis Kit, SIGMA with a molecular cutoff: 6-8 kDa), sealed, and put into a 50 mL conical tube filled with 20 mL of PBS with a composition of 0.1% Tween 20 aqueous solution. The tube was then placed on a 37° C. shaker and rotated at 150 rpm. At the following scheduled intervals (0 minutes, 5 minutes, 10 minutes, 15 minutes, 30 minutes, 60 minutes, 2 hours, 4 hours, 8 hours, 1 day, 2 days, 4 days, 6 days, 8 days, 10 days, 12 days, and 14 days), 200 μl of the release medium was collected for liquid chromatography-tandem mass spectrometer (LC-MS/MS). immediately after that, 200 μl of fresh dissolution medium (PBS) at 37° C. was replenished. Samples were collected as triplicates. The exact concentration of FD-TFV was used as a control. TFV was monitored by LC-MS/MS using an AB Sciex (Framingham, MA) 6500+QTRAP® mass spectrometer coupled to a Shimadzu (Columbia, MD) NexeraX2 LC. TFV was measured with the mass spectrometer in positive MRM (multiple reaction monitoring) modes by following the precursor to fragment ion transitions 288.1 to 176.2. A Kinetex C8 column (5 micron, 100×4.6 mm) was used for chromatography with the following conditions: Buffer A: dH20+0.1% formic acid, Buffer B: acetonitrile+0.1% formic acid, 0-1.0 min 5% B, 1.0-3.0 min gradient to 100% B, 3.0-5.0 min 100% B, 5.0-5.1 min gradient to 5% B, 5.1-6.0 5% B. Indinavir (transition 614.6 to 138.9) was used as an internal standard (IS). 50 μl of the provided sample was mixed with 100 μl of 50:50 methanol:0.02N HCL containing 50 ng/mL Indinavir IS. Samples were vortexed for 15 sec, incubated at room temp for 10′, and spun at 16,100×g 4° C. in a refrigerated microcentrifuge. LC-MS/MS then evaluated the supernatant. Standard curves were generated using PBS spiked with varying concentrations of TFV or TDF and processed as described above. The concentration of drug in each timepoint sample was quantified using Analyst 1.7 software (AB Sciex). A value of 3-fold above the signal obtained from blank PBS was designated the limit of detection (LOD). The limit of quantitation (LOQ) was defined as the lowest concentration at which back-calculation yielded a concentration within 20% of theoretical and above the LOD.

Drug release of ND in vitro environment through the BBB: Primary Human Brain Microvascular Endothelial Cells (HBMVEC) and human astrocytes (HA) (ScienCell Research Laboratories, Carlsbad, CA) were obtained and prepared for plating of the BBB model. In vitro BBB model was established in a transwell plate. HA was split and seeded on the lower side of a 0.4 um pore size PTFE membrane tissue culture inserts at an initial concentration of 105 cells per well. HBMVEC was incubated for 2 hours to allow cells to be saturated on the outside of the insert. After incubation, a confluent layer of HBMVEC was grown on the upper side of the membrane. Following 24 hours of incubation, the BBB's integrity was measured with transendothelial electrical resistance (TEER) using Millicell ERS microelectrodes. Typical TEER values of untreated BBB were observed to be around ˜140 Ω/cm². Cells were allowed to grow up to 70% confluency, and the pre-determined concentration of ND was introduced into the upper chamber of the transwell insert. Following the introduction to the upper chamber, media was collected at various time points (30 minutes, 1 hour, 1 day, 2 days, 4 days, 6 days, 8 days, and 10 days) from the lower chamber and replenished fresh media. Samples were taken as triplicates for each treatment. The samples were stored at −20° C. until further analyses. The drug content from the collected media samples with same conditions as forementioned above. Additionally, TEER measurements were performed at 24 hours post-treatment and at each time point collection. Simultaneously, a separate set up with the same concentration of FD-TFV was used as a control.

Cell uptake and characterization of extended-release from ND: Microglial uptake, retention, and release of 1:20 ND, 1:4 ND, and FD-TFV were determined. HMC-3 cells were cultured in 24-well at 50,000 cells per well and incubated at 37° C. in a humidified, 5% CO2 atmosphere for 24 hours to allow for 70% confluence. After 24 hours of cell growth, samples were assigned into different treatment groups of 1:20 ND, 1:4 ND, and FD-TFV and treated at 0.0625 mg/mL concentration. Following drug treatment, samples were incubated at 37° C., and cell uptake was determined every hour over 8 hours and at a 24-hour timepoint. For each sample, media was collected into microcentrifuge tubes and stored at −20° C. for later analysis. Samples were taken as triplicates for each treatment. Cells were trypinized and centrifuged to collect pellets. Cell pellets were then stored at −20° C. for later analysis.

Cell pellets were washed in 1 mL of PBS and centrifuged at 3,000 rpm for 8 minutes at 4° C. PBS was then discarded, and cell pellets were resuspended in 200 uL of HPLC-grade methanol, homogenized, and centrifuged at 14,000 rpm for 10 minutes at 4° C. The methanol extract was then collected into a separate microcentrifuge tube, and cell debris/pellet was discarded. Samples were then placed into a speed vac at 60° C. (Eppendorf Vacufuge Plus, Hauppauge, NY) to dry out samples. Samples were then stored at −20° C. for later analysis and resuspended with 50 uL of PBS for analysis.

In vivo maximum tolerated dose study and pharmacokinetic analysis of ND: Eight-week-old, healthy BALB/c mice (1:1 male and female) were purchased from Charles River Laboratories (Hollister, CA) and housed under a 12-hour/12-hour light/dark cycle. An Envigo Teklad Certified Global 18% protein rodent diet, #2018C, and water were provided to the mice ad libitum. Mice were administered a single dose intravenous (iv) dose administration. The average weight of Phase A and Phase B mice were 17.3-18.6 g and 17.2-23.4 g, respectively. All procedures were per the current Association for Assessment and Accreditation of Laboratory Animal Care (AAALAC) recommendations. In vivo studies were carried out in collaboration with NIH-DAIDS Preclinical Contract Services. This in vivo study was set up into two phases: Phase A was performed to determine the maximum tolerated dose (MTD study), and Phase B was performed to determine the plasma pharmacokinetics (PK study) of two formulations of TNF (ND and a saline preparation) following an iv dose administration.

TABLE 4
MTD Study groups and their associated treatments.

| | | | Dose | Dose | Dosing | | Drug to |
|---|---|---|---|---|---|---|---|
| | | Dose | Level | Conc. | Volume | No. of | Lipid |
| Group ^{a} | Treatment | Route | (mg/kg) | (mg/ml) | (ml/kg) | Animals | Ratio ^{b} |
| 1 | Bicelle ^{c} | iv | 0 | 0 | 4 | 2M/2F | 0 |
| 2 | tenofovir | iv | 2 | 0.95 | 2.1 | 2M/2F | 1:20 |
| 3 | tenofovir | iv | 10 | 2.50 | 4 | 2M/2F | 1:7.3 |
| 4 | tenofovir | iv | 15 | 3.75 | 4 | 2M/2F | 1:5 |
| 5 | tenofovir | iv | 20 | 5.00 | 4 | 2M/2F | 1:3.8 |

^{a} All animals were treated on Day 1 and observed for up to 48 hr. There was a 30-45 min time interval between each dose group to observe the treated animals. Surviving mice from each dose group were euthanized ~48 hr postdose: necropsies were not performed on the mice.
^{b} Each dose group had a lipid concentration of 5%.
^{c} Bicelle = lipid: 5 mg/100 μl

For the MTD study, ten males and ten females used for this phase were divided into five different treatment groups (Table 4). There were 30-45 minutes time intervals between the dose groups, and mice were observed immediately postdose, 30-45 minutes postdose, and once daily up to 48 hours for toxicity signs. Animals were monitored for any altered clinical signs such as gross motor and behavioral activity and any observable appearance changes.

TABLE 5
PK study groups and their associated treatments (ND and FD-TFV).

| | | | Dose | Dose | Dosing | | Blood |
|---|---|---|---|---|---|---|---|
| | | Dose | Level | Conc. | Volume | No. of | Collection |
| Group | Treatment ^{a} | Route | (mg/kg) ^{b} | (mg/ml) ^{b} | (ml/kg) | Animals ^{c} | Times (hr) ^{d} |
| 6 | Tenofovir | iv | 20 | 5 | 4 | 12M/12F | 0.167, 0.5, |
| | (in lipid | | | | | | 1, 3, 8, 24, |
| | formulation) | | | | | | 48 and 72 |
| 7 | Tenofovir (in | iv | 20 | 5 | 4 | 12M/12F | 0.167, 0.5, |
| | sterile saline | | | | | | 1, 3, 8, 24, |
| | formulation) | | | | | | 48 and 72 |

^{a} Two different dose formulations were used in Phase B.
^{b} Dose level and dose concentration was selected based on the MTD (Phase A) results and in consultation with the Sponsor.
^{c} Blood was collected from 3 untreated mice per gender for baseline control samples.
^{d} Two blood samples were collected from each mouse; the last sample was a terminal bled; three mice per gender were assigned for each time point. Blood was processed to plasma.

During the PK study, 24 males and 24 females used for this phase were divided into two groups (Table 5). Two-dose formulations of TNF were used in this phase as a single iv dose administration; the ND supplied at 5 mg/mL (Group 6) and a freshly prepared TNF saline solution at 5 mg/mL (Group 7). Additionally, six untreated mice were used to compare as baseline samples. Blood was collected for drug plasma levels at 0.167, 0.5, 1, 3, 8, 24, 48, and 72 hours postdose. Animals were observed immediately postdose, once daily, and before the last blood collection. PK data analysis was performed using the plasma concentrations of TNF via LC-MS/MS.

All reagents used in the plasma samples analysis were either HPLC-grade or American Chemical Society (ACS) reagent grade. CD-1 mouse plasma collected with K3 EDTA anticoagulant was purchased from BioIVT (Westbury, NY). The test article Tenofovir (the monohydrate form) was supplied by US Pharmacopeia. Medical Isotopes, Inc supplied the standard internal Tenofovir-d6, and the purity was assumed to be 100% during stock solution preparation. Calibration standards, quality control samples, blank plasma samples, and the study samples were placed into microcentrifuge tubes and spiked with an internal standard spiking solution (except the blank plasma samples). Samples were briefly vortexed before centrifugation. Following centrifugation, the supernatant was transferred into glass autosampler vials containing Milli-Q water and briefly vortexed. Samples were then stored into a refrigerated autosampler (set at 5° C.) before injection into the LC-MS/MS system.

TFV was detected with the mass spectrometer in positive MRM (multiple reaction monitoring) mode by following the precursor to fragment ion transitions 288.1 to 176.2 for TFV. A Phenomenex Synergi Polar-RP column (4 microns, 100×2 mm) was used for chromatography with the following conditions: Buffer A: 2% acetic acid in water, Buffer B: 0.1% acetic acid in acetonitrile, 0-2.0 min 2% B, 2.0-2.01 min gradient to 98% B, 2.01-3.5 min gradient to 98% B, and then 3.51-5 min gradient to 2% B. The concentration of drug in each timepoint sample was quantified using Analyst 1.7 software (AB Sciex).

Statistical Analysis: Experiments were performed in multiple replicates with data presented as mean±SEM. Each experiment's statistical significance was analyzed by one-way ANOVA or two-way ANOVA test with posthoc Dunnett's multiple comparisons test by GraphPad Prism software (GraphPad Prism Software Inc., San Diego, CA). P-values of ≤0.05 were considered significant.

### Example 3— Targeted Nanodrugs

Nanodrugs are developed comprising nanodiscs encapsulating an anti-HIV drug conjugated with the brain resident macrophage microglia-specific antibody Siglec-H on the surface. As microglia are a major contributor to HIV-1 infection and inflammation in the brain, the nanodrug-based anti-HIV drug is delivered specifically to microglia.

The 200 μL of nanodisc aqueous solution is incubated with 20 μL of N-(3-dimethylaminopropyl)-N′-ethylcarbodiimide hydrochloride solution (1.92 mg/mL in water, 1.04×10-6 mol) for 5-10 minutes, and then, 14 μL solution of antibodies (0.5 mg/mL, 4.67×10-11 mol) are added. After 1.5 hours of incubation, the antibody-conjugated nanodiscs are spun down at 10,000 rpm for 10 minutes and then redispersed in 220 μL of phosphate-buffered saline (PBS) for further application.

From the site of injection, the nanodrug may be taken up by the circulating monocytes and travel towards the brain as a natural immune response to neuroinflammation. While inside the brain, the macrophages reach to the site of inflammation where the majority of infected microglia are present. At the site, the infiltrating monocytes/macrophages release their payload through Trojan horse mechanisms. The nanodrug then binds with microglia specific membrane protein Tmem19 or Siglec-H and releases the drug slowly onsite. The sustained release of drug can directly act on HIV from activated microglia and reduce the inflammation for a longer period.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aresh, et al., Nanotechnol. 12, 1852-1863, 2016.
Best B M, et al. AIDS. 23:83-87, 2009.
Iqbal et al., Nanotechnology, 22(10):195102, 2011.
Liu, et al. Methods Mol Biol. 1522:273-282, 2017.
Mahabir, et al., Biochim Biophys Acta, 1828(3): p. 1025-35, 2013.
Nieh, et al., Phys Rev Lett, 91(15): p. 158105, 2003.
Nieh, et al., Methods Enzymol., 465: p. 3-20, 2009.
Nieh, et al., Langmuir, 27(23): p. 14308-16, 2011.
Nieh, et al., Langmuir, 2005. 21 (15): p. 6656-61, 2005.
Overall et al. J of Neuro Virol. 15. 360-370, 2009.
Roy et al. PLoS One. 8(10):e75374, 2013.
Sarkar, et al. J Nanosci Nanotechnol. 14(1):730-43, 2014.
Suri et al. J Occup Med Toxicol. 2:16, 2007.
Tahmasbi, et al. ACS Appl Mater Interfaces. 11(11):10505-10519, 2019.
U.S. Patent Publication No. US20170143842
Varatharajan and Thomas. Antiviral Res. 84(2):203, 2009.
Walsh, et al. Retrovirology. 11:35, 2014.
WHO ‘Consolidated guidelines on the use of antiretroviral drugs for treating and preventing HIV infection, 2013.

What is claimed is:

1. A targeted nanodrug composition comprising nanocarriers encapsulating a therapeutic agent, wherein the nanocarriers are conjugated to a targeting antibody or fragment thereof.

2. The composition of claim 1, wherein the nanocarriers are nanodiscs and/or liposomes.

3. The composition of claim 1, wherein the nanocarriers are nanodiscs.

4. The composition of claim 1, wherein the nanocarriers are liposomes.

5. The composition of claim 1, wherein the nanocarriers are nanodiscs and liposomes.

6. The composition of any of claims 2-5, wherein the nanodiscs comprise a mixture of at least long-chain phospholipid and at least one short-chain phospholipid.

7. The composition of claim 6, wherein the at least one long-chain phospholipid is selected from the group consisting of dipalmitoyl phosphocholine (DPPC), dipalmitoyl phosphatidylglycerol (DPPG), dimyristoyl phosphatidylcholine (DMPC), dioleoylphosphatidylserine (DOPS), dioleoylphosphatidylglycerol (DOPG), dioleoylphosphatidylinositol (DOPI), dioleoylphosphatidic acid (DOPA), or a mixture thereof.

8. The composition of claim 6, wherein the at least one long-chain phospholipid is DPPC or DMPC.

9. The composition of any of claims 6-8, wherein the at least one short-chain phospholipid is dihexanoyl phosphatidylcholine (DHPC).

10. The composition of any of claims 2-9, wherein the nanodiscs further comprise an anionic phospholipid.

11. The composition of claim 10, wherein the anionic phospholipid is 1,2-Dimyristoyl-sn-glycero-3-phosphoglycerol (DMPG).

12. The composition of any of claims 2-11, wherein the nanodiscs further comprise polyethylene glycol (PEG).

13. The composition of any of claims 2-11, wherein the nanodiscs further comprise polyethylene glycol (PEG2000)-conjugated distearoyl phosphoethanolamine (DSPE-PEG2000).

14. The composition of any of claims 2-11, wherein the nanodiscs comprise [DPPG]/[DPPC] at a molar ratio of 0.01 to 0.1.

15. The composition of any of claims 2-14, wherein the nanodiscs comprise ([DPPC]+[DPPG])/[DHPC] at a molar ratio of 2 to 5.

16. The composition of any of claims 2-14, wherein the nanodiscs comprise DPPC, DHPC, DPPG, and DSPE-PEG2000.

17. The composition of any of claims 2-15, wherein the nanodiscs have a diameter of 30-40 nm.

18. The composition of claim 17, wherein the nanodiscs have a thickness of about 5 nm.

19. The composition of any of claims 2-18, wherein the nanodiscs have a hydrodynamic radius of 8-15 nm.

20. The composition of any of claims 2-18, wherein the nanodiscs have a hydrodynamic radius of 10-13 nm.

21. The composition of any of claims 2-20, wherein the liposomes have a hydrodynamic radius of 300 nm to 500 nm.

22. The composition of any of claims 2-21, wherein the liposomes have a hydrodynamic radius of about 400 nm.

23. The composition of any of claims 2-22, wherein the liposomes have a lipid charge density of 1% to 10%.

24. The composition of any of claims 2-23, wherein the liposomes have a lipid charge density of 2% to 5%.

25. The composition of any of claims 2-24, wherein the liposomes have a lipid charge density of 2% or 5%.

26. The composition of any of claims 2-25, wherein the liposomes comprise dipalmitoyl phosphocholine (DPPC), dipalmitoyl phosphatidylglycerol (DPPG), dimyristoyl phosphatidylcholine (DMPC), dioleoylphosphatidylserine (DOPS), dioleoylphosphatidylglycerol (DOPG), dioleoylphosphatidylinositol (DOPI), dioleoylphosphatidic acid (DOPA), dihexanoyl phosphatidylcholine (DHPC), or a mixture thereof.

27. The composition of any of claims 2-26, wherein the liposomes comprise DPPC and DPPG.

28. The composition of any of claims 1-27, wherein the therapeutic agent is at least one anti-retroviral therapy.

29. The composition of claim 28, wherein the at least one anti-retroviral therapy comprises tenofovir, efavirenz, lopinavir, ritonavir, emtricitabine, rilpivirine, tenofovir disoproxil fumarate, elvitegravir, cobicistat, elvitegravir, doravirine, lamivudine, dolutegravir, rilpivirine, bictegravir, atazanavir, abacavir, fostemsavir, raltegravir, maraviroc, enfuvirtide, enfuvirtide, tipranavir, fosamprenavir, darunavir, rilpivirine, nevirapine, etravirine, doravirine, or dultagravir.

30. The composition of claim 28, wherein the at least one anti-retroviral therapy is a protease inhibitor or reverse transcriptase inhibitor.

31. The composition of claim 30, wherein the protease inhibitor is indinavir sulfate ethanolate, saquinavir, saquinavir mesylate, ritonavir, nelfinavir mesylate, lopinavir, amprenavir, or atazanavir.

32. The composition of claim 30, wherein the reverse transcriptase inhibitor is tenofovir, zidovudine, lamivudine, sanilvudine, didanosine, abacavir sulfate, nevirapine, efavirenz, or delavirdine mesylate.

33. The composition of claim 28, wherein the at least one anti-retroviral therapy does not comprise efavirenz.

34. The composition of any of claims 1-33, wherein the nanodrug comprises a therapeutic agent to lipid ratio of 1:10 to 1:1000.

35. The composition of any of claims 1-33, wherein the nanodrug comprises a therapeutic agent to lipid ratio of 1:2 to 1:20.

36. The composition of any of claims 1-33, wherein the nanodrug comprises a therapeutic agent to lipid ratio of 1:4 or 1:20.

37. The composition of any of claims 1-33, wherein the nanodrug comprises a therapeutic agent to lipid ratio of 1:3.

38. The composition of any of claims 1-33, wherein the targeting antibody or fragment thereof allows delivery to the brain, lymph nodes, and/or gut-associated lymphoid tissue.

39. The composition of any of claims 1-38, wherein the targeting antibody or fragment thereof allows delivery across the blood brain barrier.

40. The composition of any of claims 1-39, wherein the targeting antibody or fragment thereof is neuron specific or microglia specific.

41. The composition of claim 40, wherein the microglia targeting antibody or fragment thereof is a Tmem19 antibody or a Siglec-H antibody.

42. The composition of any of claims 1-38, wherein the targeting antibody or fragment thereof is anti-EGFR monoclonal antibody, or anti-IGFBP7 sdAb.

43. The composition of any of claims 1-41, wherein the nanodrug is PEGylated.

44. A pharmaceutical composition comprising a plurality of targeted nanodrugs of any one of claims 1-43 in combination with a pharmaceutically acceptable carrier.

45. A method of delivering a therapeutic agent into a cell comprising administering an effective amount of targeted nanodrugs of any one of claims 1-43 to the cell.

46. A method of treating a disease or disorder in subject in need thereof comprising administering an effective amount of targeted nanodrugs of any one of claims 1-43 to the subject.

47. The method of claim 46, wherein the subject is positive for human immunodeficiency virus (HIV).

48. The method of claim 46 or 47, wherein the targeted nanodrugs are administered orally, topically, intravenously, intraperitoneally, intramuscularly, endoscopically, percutaneously, subcutaneously, regionally, or by direct injection.

49. The method of any of claims 46-48, wherein the subject is human.

50. The method of any of claims 46-49, wherein the subject has been previously treated with anti-retroviral therapy.

51. The method of any of claims 46-50, further comprising administering an additional therapeutic agent.

52. A pharmaceutical composition comprising targeted nanodrugs of any of claims 1-43 for use in the treatment of a disease or disorder in a subject.

53. The composition of claim 52, wherein the subject is positive for HIV.
